SEQUENCE LISTING (8 sequences)

SEQ ID NO: 1 - DNA, 1140 bp, synthetic construct
  source 1..1140: mol_type = other DNA
atggagacagacacactcctgctatgggtactgctgctctgggttccaggttccactggtgatatccaactagtagaaagcggcggcggcctagtagaaccaggcggcagcctacgactaagctgcgcagcaagcggcataatattcaaaataaacgacatgggctggtaccgacaagcaccaggcaaacgacgagaatgggtagcagcaagcacaggcggcgacgaagcaatataccgagacagcgtaaaagaccgattcacaataagccgagacgcaaaaaacagcgtattcctacaaatgaacagcctaaaaccagaagacacagcagtatactactgcacagcagtaataagcacagaccgagacggcacagaatggcgacgatactggggccaaggcacacaagtaacagtaagcagcgaagcggccgcaaaagaagcagcggcaaaagaagcggcagcaagatcttcaggacgtacgatcgagttcatgtacccccctccctacctggacaacgagagaagcaacggcaccatcatccacatcaaagaaaagcacctgtgccacacccagagcagccccaagctgttctgggccctggtggtggtggccggcgtgctgttctgttacggcctgctggtcacagtggccctgtgcgtgatctggaccaacagcagaagaaacagaggcggccagagcgactacatgaacatgacccccagaaggccaggcctgaccagaaagccctaccagccctacgcccctgccagagacttcgccgcctacagaccggttagagccaagttcagcagatccgccgagacagccgccaacctgcaggatcccaaccagctgtacaacgagctgaacctgggcagacgggaggaatacgacgtgctggaaaagaagagagccagggaccccgagatgggcggcaagcagcagagaagaagaaaccctcaggaaggcgtctacaacgccctgcagaaagacaagatggccgaggcctacagcgagatcggcaccaagggcgagagaagaaggggcaagggccacgatggcctgtaccagggcctgtccaccgccaccaaggacacctacgacgccctgcacatgcagaccctggcccccagatga

SEQ ID NO: 2 - DNA, 71 bp, synthetic construct
  source 1..71: mol_type = other DNA
aggagacccaagctggctagggtggcgggttctctctgagtctgtggggaccagaagaatactagtgccac

SEQ ID NO: 3 - DNA, 292 bp, synthetic construct
  source 1..292: mol_type = other DNA
ctcgagctggtactgcatgcacgcaatgctagatgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccccggccgc

SEQ ID NO: 4 - DNA, 1656 bp, Mus musculus
  source 1..1656: mol_type = genomic DNA
atggcattgcccgtgactgctcttctgctcccacttgccctcctgcttcacgctgcacgaccaagtcaagggcgcgccatgtgggagctggagaaagacgtttatgttgtagaggtggactggactcccgatgcccctggagaaacagtgaacctcacctgtgacacgcctgaagaagatgacatcacctggacctcagaccagagacatggagtcataggctctggaaagaccctgaccatcactgtcaaagagtttctagatgctggccagtacacctgccacaaaggaggcgagactctgagccactcacatctgctgctccacaagaaggaaaatggaatttggtccactgaaattttaaaaaatttcaaaaacaagactttcctgaagtgtgaagcaccaaattactccggacggttcacgtgctcatggctggtgcaaagaaacatggacttgaagttcaacatcaagagcagtagcagttcccctgactctcgggcagtgacatgtggaatggcgtctctgtctgcagagaaggtcacactggaccaaagggactatgagaagtattcagtgtcctgccaggaggatgtcacctgcccaactgccgaggagaccctgcccattgaactggcgttggaagcacggcagcagaataaatatgagaactacagcaccagcttcttcatcagggacatcatcaaaccagacccgcccaagaacttgcagatgaagcctttgaagaactcacaggtggaggtcagctgggagtaccctgactcctggagcactccccattcctacttctccctcaagttctttgttcgaatccagcgcaagaaagaaaagatgaaggagacagaggaggggtgtaaccagaaaggtgcgttcctcgtagagaagacatctaccgaagtccaatgcaaaggcgggaatgtctgcgtgcaagctcaggatcgctattacaattcctcatgcagcaagtgggcatgtgttccctgcagggtccgatccggcggcggcgggagtggcggcgggggttctggcggaggcctcgctagcggtggctccagggtcattccagtctctggacctgccaggtgtcttagccagtcccgaaacctgctgaagaccacagatgacatggtgaagacggccagagaaaaactgaaacattattcctgcactgctgaagacatcgatcatgaagacatcacacgggaccaaaccagcacattgaagacctgtttaccactggaactacacaagaacgagagttgcctggctactagagagacttcttccacaacaagagggagctgcctgcccccacagaagacgtctttgatgatgaccctgtgccttggtagcatctatgaggacttgaagatgtaccagacagagttccaggccatcaacgcagcacttcagaatcacaaccatcagcagatcattctagacaagggcatgctggtggccatcgatgagctgatgcagtctctgaatcataatggcgagactctgcgccagaaacctcctgtgggagaagcagacccttacagagtgaaaatgaagctctgcatcctgcttcacgccttcagcacccgcgtcgtgaccatcaacagggtgatgggctatctgagctccgcctga

SEQ ID NO: 5 - RNA, 1140 nt, synthetic construct
  source 1..1140: mol_type = other RNA
  misc_difference 1..1140: note = N1-Methylpseudo-UTP
anggagacagacacacnccngcnangggnacngcngcncngggnnccaggnnccacnggngananccaacnagnagaaagcggcggcggccnagnagaaccaggcggcagccnacgacnaagcngcgcagcaagcggcanaananncaaaanaaacgacangggcnggnaccgacaagcaccaggcaaacgacgagaangggnagcagcaagcacaggcggcgacgaagcaananaccgagacagcgnaaaagaccganncacaanaagccgagacgcaaaaaacagcgnannccnacaaangaacagccnaaaaccagaagacacagcagnanacnacngcacagcagnaanaagcacagaccgagacggcacagaanggcgacganacnggggccaaggcacacaagnaacagnaagcagcgaagcggccgcaaaagaagcagcggcaaaagaagcggcagcaagancnncaggacgnacgancgagnncangnaccccccncccnaccnggacaacgagagaagcaacggcaccancanccacancaaagaaaagcaccngngccacacccagagcagccccaagcngnncngggcccnggnggnggnggccggcgngcngnncngnnacggccngcnggncacagnggcccngngcgngancnggaccaacagcagaagaaacagaggcggccagagcgacnacangaacangacccccagaaggccaggccngaccagaaagcccnaccagcccnacgccccngccagagacnncgccgccnacagaccggnnagagccaagnncagcaganccgccgagacagccgccaaccngcaggancccaaccagcngnacaacgagcngaaccngggcagacgggaggaanacgacgngcnggaaaagaagagagccagggaccccgagangggcggcaagcagcagagaagaagaaacccncaggaaggcgncnacaacgcccngcagaaagacaaganggccgaggccnacagcgagancggcaccaagggcgagagaagaaggggcaagggccacganggccngnaccagggccngnccaccgccaccaaggacaccnacgacgcccngcacangcagacccnggcccccaganga

SEQ ID NO: 6 - RNA, 1656 nt, synthetic construct
  source 1..1656: mol_type = other RNA
  misc_difference 1..1656: note = N1-Methylpseudo-UTP
anggcanngcccgngacngcncnncngcncccacnngcccnccngcnncacgcngcacgaccaagncaagggcgcgccangngggagcnggagaaagacgnnnangnngnagaggnggacnggacncccgangccccnggagaaacagngaaccncaccngngacacgccngaagaagangacancaccnggaccncagaccagagacanggagncanaggcncnggaaagacccngaccancacngncaaagagnnncnagangcnggccagnacaccngccacaaaggaggcgagacncngagccacncacancngcngcnccacaagaaggaaaanggaannnggnccacngaaannnnaaaaaannncaaaaacaagacnnnccngaagngngaagcaccaaannacnccggacggnncacgngcncanggcnggngcaaagaaacanggacnngaagnncaacancaagagcagnagcagnnccccngacncncgggcagngacangnggaanggcgncncngncngcagagaaggncacacnggaccaaagggacnangagaagnanncagngnccngccaggaggangncaccngcccaacngccgaggagacccngcccanngaacnggcgnnggaagcacggcagcagaanaaanangagaacnacagcaccagcnncnncancagggacancancaaaccagacccgcccaagaacnngcagangaagccnnngaagaacncacaggnggaggncagcngggagnacccngacnccnggagcacnccccannccnacnncncccncaagnncnnngnncgaanccagcgcaagaaagaaaagangaaggagacagaggaggggngnaaccagaaaggngcgnnccncgnagagaagacancnaccgaagnccaangcaaaggcgggaangncngcgngcaagcncaggancgcnannacaannccncangcagcaagngggcangngnncccngcagggnccganccggcggcggcgggagnggcggcgggggnncnggcggaggccncgcnagcggnggcnccagggncannccagncncnggaccngccaggngncnnagccagncccgaaaccngcngaagaccacagangacanggngaagacggccagagaaaaacngaaacannannccngcacngcngaagacancgancangaagacancacacgggaccaaaccagcacanngaagaccngnnnaccacnggaacnacacaagaacgagagnngccnggcnacnagagagacnncnnccacaacaagagggagcngccngcccccacagaagacgncnnngangangacccngngccnnggnagcancnangaggacnngaagangnaccagacagagnnccaggccancaacgcagcacnncagaancacaaccancagcagancanncnagacaagggcangcnggnggccancgangagcngangcagncncngaancanaanggcgagacncngcgccagaaaccnccngngggagaagcagacccnnacagagngaaaangaagcncngcanccngcnncacgccnncagcacccgcgncgngaccancaacagggngangggcnancngagcnccgccnga

SEQ ID NO: 7 - RNA, 1140 nt, synthetic construct
  source 1..1140: mol_type = other RNA
atggagacagacacactcctgctatgggtactgctgctctgggttccaggttccactggtgatatccaactagtagaaagcggcggcggcctagtagaaccaggcggcagcctacgactaagctgcgcagcaagcggcataatattcaaaataaacgacatgggctggtaccgacaagcaccaggcaaacgacgagaatgggtagcagcaagcacaggcggcgacgaagcaatataccgagacagcgtaaaagaccgattcacaataagccgagacgcaaaaaacagcgtattcctacaaatgaacagcctaaaaccagaagacacagcagtatactactgcacagcagtaataagcacagaccgagacggcacagaatggcgacgatactggggccaaggcacacaagtaacagtaagcagcgaagcggccgcaaaagaagcagcggcaaaagaagcggcagcaagatcttcaggacgtacgatcgagttcatgtacccccctccctacctggacaacgagagaagcaacggcaccatcatccacatcaaagaaaagcacctgtgccacacccagagcagccccaagctgttctgggccctggtggtggtggccggcgtgctgttctgttacggcctgctggtcacagtggccctgtgcgtgatctggaccaacagcagaagaaacagaggcggccagagcgactacatgaacatgacccccagaaggccaggcctgaccagaaagccctaccagccctacgcccctgccagagacttcgccgcctacagaccggttagagccaagttcagcagatccgccgagacagccgccaacctgcaggatcccaaccagctgtacaacgagctgaacctgggcagacgggaggaatacgacgtgctggaaaagaagagagccagggaccccgagatgggcggcaagcagcagagaagaagaaaccctcaggaaggcgtctacaacgccctgcagaaagacaagatggccgaggcctacagcgagatcggcaccaagggcgagagaagaaggggcaagggccacgatggcctgtaccagggcctgtccaccgccaccaaggacacctacgacgccctgcacatgcagaccctggcccccagatga

SEQ ID NO: 8 - RNA, 1656 nt, synthetic construct
  source 1..1656: mol_type = other RNA
atggcattgcccgtgactgctcttctgctcccacttgccctcctgcttcacgctgcacgaccaagtcaagggcgcgccatgtgggagctggagaaagacgtttatgttgtagaggtggactggactcccgatgcccctggagaaacagtgaacctcacctgtgacacgcctgaagaagatgacatcacctggacctcagaccagagacatggagtcataggctctggaaagaccctgaccatcactgtcaaagagtttctagatgctggccagtacacctgccacaaaggaggcgagactctgagccactcacatctgctgctccacaagaaggaaaatggaatttggtccactgaaattttaaaaaatttcaaaaacaagactttcctgaagtgtgaagcaccaaattactccggacggttcacgtgctcatggctggtgcaaagaaacatggacttgaagttcaacatcaagagcagtagcagttcccctgactctcgggcagtgacatgtggaatggcgtctctgtctgcagagaaggtcacactggaccaaagggactatgagaagtattcagtgtcctgccaggaggatgtcacctgcccaactgccgaggagaccctgcccattgaactggcgttggaagcacggcagcagaataaatatgagaactacagcaccagcttcttcatcagggacatcatcaaaccagacccgcccaagaacttgcagatgaagcctttgaagaactcacaggtggaggtcagctgggagtaccctgactcctggagcactccccattcctacttctccctcaagttctttgttcgaatccagcgcaagaaagaaaagatgaaggagacagaggaggggtgtaaccagaaaggtgcgttcctcgtagagaagacatctaccgaagtccaatgcaaaggcgggaatgtctgcgtgcaagctcaggatcgctattacaattcctcatgcagcaagtgggcatgtgttccctgcagggtccgatccggcggcggcgggagtggcggcgggggttctggcggaggcctcgctagcggtggctccagggtcattccagtctctggacctgccaggtgtcttagccagtcccgaaacctgctgaagaccacagatgacatggtgaagacggccagagaaaaactgaaacattattcctgcactgctgaagacatcgatcatgaagacatcacacgggaccaaaccagcacattgaagacctgtttaccactggaactacacaagaacgagagttgcctggctactagagagacttcttccacaacaagagggagctgcctgcccccacagaagacgtctttgatgatgaccctgtgccttggtagcatctatgaggacttgaagatgtaccagacagagttccaggccatcaacgcagcacttcagaatcacaaccatcagcagatcattctagacaagggcatgctggtggccatcgatgagctgatgcagtctctgaatcataatggcgagactctgcgccagaaacctcctgtgggagaagcagacccttacagagtgaaaatgaagctctgcatcctgcttcacgccttcagcacccgcgtcgtgaccatcaacagggtgatgggctatctgagctccgcctga

(19) United States
(12) Patent Application Publication
(10) Pub. No.: US 2026/0144810 A1
(43) Pub. Date: May 28, 2026

(54) mRNA COMPOSITION FOR TREATING CANCER, PREPARATION CONTAINING THE SAME AND USE THEREOF

(71) Applicant: INDUSTRIAL TECHNOLOGY RESEARCH INSTITUTE, Hsinchu (TW)

(72) Inventors: Min-Yuan CHOU, Taipei City (TW); Yen-Ju LIN, Zhudong Township (TW); Ming-Hua YANG, Zhubei City (TW); Ya-Ping LAI, Zhudong Township (TW); Mei-Wei LIN, Zhubei City (TW); Li-Tsen LIN, New Taipei City (TW); Yu-Jia TZENG, Zhubei City (TW); Ssu-Yuan WU, Zhubei City (TW); Chin Pen LAI, Zhubei City (TW)

(73) Assignee: INDUSTRIAL TECHNOLOGY RESEARCH INSTITUTE, Hsinchu (TW)

(21) Appl. No.: 19/214,449

(22) Filed: May 21, 2025

Related U.S. Application Data
(60) Provisional application No. 63/725,841, filed on Nov. 27, 2024.

(51) Int. Cl. A61K 31/7105; A61K 9/1271; A61K 9/51; A61K 38/00; A61K 40/31; A61K 40/42; A61P 35/04; C07K 14/54; C07K 14/705; C07K 14/725; C07K 16/28; C12N 15/88

(52) U.S. Cl. CPC A61K 31/7105; A61K 9/1271; A61K 9/5123; A61K 40/31; A61K 40/421; A61P 35/04; C07K 14/5434; C07K 14/7051; C07K 14/70521; C07K 16/2803; C12N 15/88; A61K 38/00; A61K 2239/13; A61K 2239/17; A61K 2239/21; A61K 2239/22; C07K 2317/569; C07K 2319/02

(57) ABSTRACT

An mRNA composition for treating cancer is provided. The mRNA composition for treating cancer includes an mRNA encoding a CD47-targeted chimeric antigen receptor (CAR) and an mRNA encoding interleukin-12 (IL-12).

### CROSS REFERENCE TO RELATED APPLICATIONS

This application claims the benefit of U.S. Provisional Application No. 63/725,841, filed on Nov. 27, 2024, the entirety of which is incorporated by reference herein.

### REFERENCE TO AN ELECTRONIC SEQUENCE LISTING

The contents of the electronic sequence listing (9044B-P240263501-US_ST26_Seq_Listing.xml; Size: 18,098 bytes; and Date of Creation: May 20, 2025) is herein incorporated by reference in its entirety.

### TECHNICAL FIELD

The present invention relates to cancer therapy, and in particular it relates to an mRNA composition for treating cancer, a preparation containing the same, and uses thereof.

### BACKGROUND

Cell-mediated immunity is an immune response that does not involve antibodies as opposed to humoral immunity. Cell-mediated immunity will activate macrophages and natural killer cells, causing them to destroy intracellular pathogens and activate antigen-specific cytotoxic T cells to release various cytokines in response to antigens.

Cancer immunotherapy is a method of treating cancer by activating the immune system. Tumor antigens that are recognized by the immune system exist on the surface of cancer cells, which is the basis of cancer immunity while tumor-associated antigens (TAA) are usually present on the surface of tumor cells and expressed in large quantities. Immunotherapy can be categorized into active immunity, passive immunity and combined immunity, wherein active immunotherapy directly induces the autoimmune system to recognize tumor antigens and then attack cancer cells.

Chimeric antigen receptor T-cell (CAR-T) therapy is also a type of cellular immunotherapy, while in this therapy, T cells become T-cells that can specifically recognize tumor-associated antigens to further destroy cancer cells by taking blood from the patient, isolating the T-cells, and ex vivo genetically engineering the T-cells, and the modified T cells are then injected back into the patient's body to replicate in large numbers and achieve the effect of cellular immunity.

However, cell therapies for cancer, such as chimeric antigen receptor T cell therapy, usually requires complex ex vivo cell engineering, which results in difficulty in operation and high costs. Therefore, currently, there is still an urgent need for a novel cell therapy for cancer therapy.

### SUMMARY

The present disclosure provides an mRNA composition for treating cancer, comprising: an mRNA encoding a CD47-targeted chimeric antigen receptor (CAR) and an mRNA encoding interleukin 12 (IL-12)

The present disclosure also provides an mRNA pharmaceutical preparation for treating cancer, comprising: the mRNA composition for treating cancer mentioned above.

The present disclosure also provides a lipid nanoparticle preparation for treating cancer, comprising: a lipid nanoparticle, an mRNA encoding a CD47-targeted chimeric antigen receptor and an mRNA encoding interleukin-12, wherein the lipid nanoparticle is loaded with the mRNA encoding a CD47-targeted chimeric antigen receptor and the mRNA encoding interleukin-12.

Moreover, the present disclosure further provides a use of an mRNA encoding a CD47-targeted chimeric antigen receptor and an mRNA encoding interleukin-12 in the manufacture of a medicament for treating cancer, wherein the medicament is capable of polarizing a M2 macrophage into a M1 macrophage to secrete interleukin-12 and form a CD47-targeted chimeric antigen receptor-macrophage (CD47-targeted CAR-Macrophage, CD47-targeted CAR-M) in a subject in need thereof.

And, the present disclosure provides a use of an mRNA encoding a CD47-targeted chimeric antigen receptor in the manufacture of a medicament for treating cancer, wherein the medicament for treating cancer is administered together with an mRNA encoding interleukin-12.

Furthermore, the present disclosure also provides a use of an mRNA encoding interleukin-12 in the manufacture of a medicament for polarizing a M2 macrophage into a M1 macrophage and/or treating cancer, wherein the medicament for treating cancer is administered together with an mRNA encoding a CD47-targeted chimeric antigen receptor.

In addition, the present disclosure provides a method for treating cancer, comprising: administering a medicament manufactured from the use of an mRNA encoding a CD47-targeted chimeric antigen receptor and an mRNA encoding interleukin-12 in the manufacture of a medicament for treating cancer of the present disclosure mentioned above, the mRNA composition for treating cancer of the present disclosure mentioned above, the pharmaceutical preparation for treating cancer of the present disclosure mentioned above or the lipid nanoparticle preparation for treating cancer of the present disclosure mentioned above to a subject in need thereof.

The present disclosure may provide another method for treating cancer, comprising: administering a medicament manufactured from the use of an mRNA encoding a CD47-targeted chimeric antigen receptor in the manufacture of a medicament for treating cancer of the present disclosure mentioned above and an mRNA encoding interleukin-12 to a subject in need thereof.

The present disclosure may provide yet another method for treating cancer, comprising: administering a medicament manufactured from the use of an mRNA encoding interleukin-12 in the manufacture of a medicament for treating cancer of the present disclosure mentioned above and an mRNA encoding a CD47-targeted chimeric antigen receptor to a subject in need thereof.

A detailed description is given in the following embodiments with reference to the accompanying drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The present invention can be more fully understood by reading the subsequent detailed description and examples with references made to the accompanying drawings, wherein:

FIG. 1 shows a schematic diagram of the rationale for IL-12/CD47-targeted CAR mRNA-LNP-mediated tumor inhibition in liver cancer;

FIG. 2 shows reverse transcription quantitative polymerase chain reaction (RT-qPCR) analysis of M1- and M2-type macrophage gene expressions following IL-12/A4-28z mRNA transfection into M2-polarized macrophage cells. M2-polarized mouse (immortalized bone marrow-derived macrophage (iBMDM) were transfected with 1 g each of IL-12 and A4-28z CAR mRNA to assess the shift in gene expression from M2 to M1 phenotype. Expression levels of M2-associated genes (Arg1, Cd206, Fizz1, and Ym1) and M1-associated genes (Il12p40, Il1b, and iNOS) were quantified by reverse transcription quantitative polymerase chain reaction. Gene expression was normalized to Gapdh as an internal control, and relative fold changes were calculated using the ^{ΔΔ}Ct method compared to M0 (non-polarized) control cells. Data are presented as mean±SD (n=3) independent experiments. Statistical significance was determined using an unpaired two-tailed t-test (** p<0.01; *** p<0.001). NT: non-transfected;

FIG. 3 shows the effect of CD47-targeted CAR mRNA-transfected macrophages on tumor cell phagocytosis potency. Flow cytometry analyses of tumor cell phagocytosis by macrophages under different conditions. Mouse immortalized bone marrow-derived macrophage as effector cells (E) were transfected with IL-12 mRNA alone or co-transfected with CD47-targeted CAR mRNAs (IL-12/A4-28z) to evaluate their phagocytic activities. Non-transfected (NT) immortalized bone marrow-derived macrophages served as the control group. Mouse CD47⁺ hepatocellular carcinoma cell line NHRI-8-B4 (target cells, T) were labeled with CMFDA (green), and immortalized bone marrow-derived macrophages were stained with CMTPX (red) to distinguish tumor cells from macrophages. Phagocytosis was assessed by identifying double-positive populations [B525 (CMFDA⁺)/V610(CMTPX⁺)] in the upper right quadrant (Q2-UR);

FIG. 4 shows florescence imaging of whole organs harvested from Ai14 mice (n=3, representative 1 pictured) 3 days after intravenous injection of Cre-LNP mRNA (left panel). Single-cell analysis of liver cells (right panel, n=2). LNP uptake and tdTomato gene expression were analyzed by flow cytometry. Specific antibody markers were used to identify hepatocytes, Kupper cells (KCs) and liver sinusoidal endothelial cells (LSECs). The percentages of tdTomato⁺ cells in each cell type are indicated;

FIG. 5A shows a schematic diagram for the mouse model of spleen-liver cancer metastasis. The mouse liver tumor (NIM-8-B4) cells carrying firefly luciferase gene were surgically implanted into the spleens of 6-8-week-old C57BL/6J mice. Tumor metastasized to liver organ was monitored using in vivo imaging system (IVIS) bioluminescence imaging to detect luminescence signals. Once splenic tumor formation was confirmed (on day 5 post-inoculation), the mice were stratified into experimental groups based on tumor progression (n=6 per group). Systemic intravenous (i.v.) administration was used for drug delivery. The experimental group received 10 μg of IL-12/A4-28z mRNA-LNP, while the control group (vehicle) received PBS as control. Injections were administered every 3 days, with a total of 3 doses. On day 3 after the final dose, mice were euthanized and their livers were collected for efficacy evaluation.

FIG. 5B shows liver images and quantification of tumor burden presented by in vivo imaging system in the mouse model of spleen-liver cancer metastasis. Tumor progression was monitored using in vivo imaging system, and representative images from each group are shown (upper panel). Quantification of tumor burden was determined by measuring luminescence signal intensity, presented as total flux (photons/sec) (lower panel);

FIG. 5C shows immunohistochemistry (IHC) staining results of CD47 expression in liver tissue in the mouse model of spleen-liver cancer metastasis. Representative immunohistochemistry staining of liver sections for CD47 was performed to evaluate the effect of mRNA-LNP treatment on tumor-associated immune evasion;

FIG. 5D shows immunohistochemistry (IHC) staining analysis of CD47 expression in liver tissue in the mouse model of spleen-liver cancer metastasis. Quantitative analysis was performed by measuring the percentage of CD47-positive cells per field (lower panel). Data are presented as mean±SD (n=3) independent experiments. Statistical significance was determined using an unpaired two-tailed t-test (*p<0.05);

FIG. 5E shows immunohistochemistry staining results of CD163⁺ macrophages in liver tissue in the mouse model of spleen-liver cancer metastasis. Liver sections were stained for CD163 (a marker of M2-like macrophages) to assess changes in the tumor-associated macrophage population;

FIG. 5F shows immunohistochemistry staining analysis of CD163⁺ macrophages in liver tissue in the mouse model of spleen-liver cancer metastasis. Representative images from each treatment group are shown, with quantification based on the percentage of CD163-positive cells in tumor-infiltrating macrophages. Data are presented as mean±SD (n=3) independent experiments. Statistical significance was determined using an unpaired two-tailed t-test (*p<0.05);

FIG. 6A shows a schematic diagram of the experimental design of the mouse model of liver metastasis. 6-8-week-old female BALB/c mice were intravenously injected with syngeneic mouse lymphoma tumor cells, A20, expressing firefly luciferase gene. The detailed experimental design and dosing regimens are described in Example 8;

FIG. 6B shows mouse images presented by in vivo imaging system in the mouse model of liver metastasis. Tumor progression and metastasis to the liver were assessed by in vivo imaging system imaging on Days 14, 21, and 25. On Day 25 (D25), mice were euthanized, and gross examination of liver was performed to quantify liver metastasis and tumor burden across experimental groups;

FIG. 6C shows quantitation of liver metastasis occurrence and tumor sizes (left panel) and liver-to-body weight ratio (right panel) in the mouse model of liver metastasis. The bar represents the mean, and each dot represents the value for an individual mouse liver. The shaded area indicates regions where no tumor nodules were detected on the liver surface. Statistical significance was determined using an unpaired two-tailed test (*p<0.05, **p<0.01, ns: not significant);

FIG. 7A shows a schematic diagram of the experimental design of another mouse model of liver metastasis. 6-8-week-old female BALB/c mice were intravenously injected with syngeneic mouse lymphoma tumor cells, A20, expressing firefly luciferase gene. The detailed experimental design and dosing regimens are described in Example 9;

FIG. 7B shows mouse images presented by in vivo imaging system in another mouse model of liver metastasis. Tumor progression and metastasis to the liver were assessed by in vivo imaging system imaging on Days 14, 21, and 25. On Day 25 (D25), mice were euthanized, and gross examination of liver was performed to quantify liver metastasis and tumor burden across experimental groups; and

FIG. 7C shows quantitation of liver metastasis occurrence and tumor sizes (left panel) and liver-to-body weight ratio (right panel) in another mouse model of liver metastasis. The bar represents the mean, and each dot represents the value for an individual mouse liver. The shaded area indicates regions where no tumor nodules were detected on the liver surface. Statistical significance was determined using an unpaired two-tailed test (*p<0.05, **p<0.01, ns: not significant).

### DETAILED DESCRIPTION

The following description is made for the purpose of illustrating the general principles of the invention and should not be taken in a limiting sense. The scope of the invention is best determined by reference to the appended claims.

The present disclosure mainly develops an mRNA composition that can form an engineered chimeric antigen receptor macrophage (CAR-M) in vivo to address the challenges of using immune cell therapy (e.g., chimeric antigen receptor macrophage, CAR-T) for solid tumor treatment.

As innate immune cells, macrophages possess several advantages for anti-tumor applications, including tumor-penetrating ability, phagocytosis, antigen presentation, and the modulation of adaptive immunity.

In the present disclosure, a novel nucleic acid composition is designed to promote M1 macrophage polarization and enable the expression of an engineered chimeric antigen receptor on macrophages, thereby enhancing tumor inhibition.

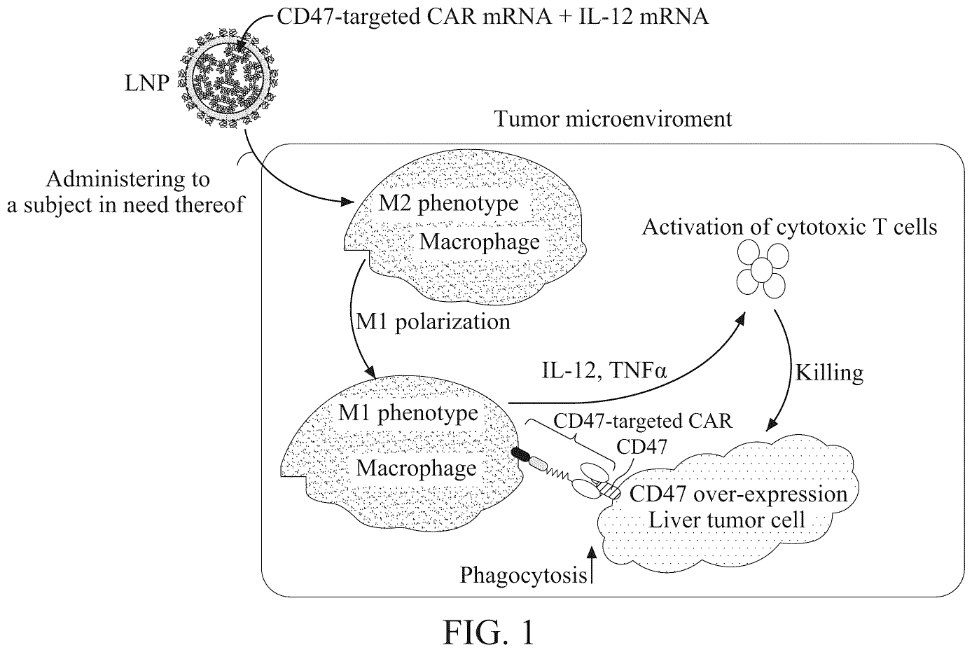

Please refer to FIG. 1, which shows a schematic diagram of the main design concepts of the present disclosure. Overall, in the present disclosure, a chimeric antigen receptor macrophage (CAR-M) cell therapy has been developed for solid tumors through the in vivo delivery of lipid nanoparticles (LNPs) encapsulating mRNA encoding a tumor-associated antigen (TAA)-specific chimeric antigen receptor. Through this innovative strategy, macrophages can be activated in vivo and made to directly express chimeric antigen receptors (CARs), thus bypassing the need for ex vivo cell engineering.

In the present disclosure, the nucleic acid composition to promote M1 macrophage polarization is interleukin-12 (IL-12) mRNA. Interleukin-12 is a pro-inflammatory cytokine primarily produced by activated dendritic cells, monocytes, macrophages and B cells (Trinchieri, G., M. Rengaraju, et al. (1993). “Producer cells of interleukin 12.” Parasitol Today 9(3): 97.). Interleukin-12 plays a critical role in bridging innate and adaptive immunity by promoting the differentiation of naïve T cells into Th1 cells which secrete interferon-gamma (IFN-γ) to enhance cellular immune responses.

Interleukin-12 also activates natural killer (NK) cells and increases their cytotoxicity, leading to enhanced destruction of infected or malignant cells. In cancer immunotherapy, interleukin-12 exhibits potent anti-tumor effects by stimulating cytotoxic T lymphocytes (CTLs) and NK cells, thereby boosting their ability to recognize and kill tumor cells (Tugues, S., S. H. Burkhard, et al. (2015). “New insights into IL-12-mediated tumor suppression.” Cell Death Differ 22(2): 237-246.). In addition, interleukin-12 can promote the production of IFN-γ, thereby enhancing antigen presentation and inhibiting angiogenesis within the tumor microenvironment, as well as limiting tumor growth and metastasis.

With regard to antitumorigenic M1 macrophages, interleukin-12 is a hallmark cytokine associated with their pro-inflammatory phenotype. Therefore, using interleukin-12 to induce the transformation of M2 macrophages into M1 polarization appears to be a promising strategy for enhancing their tumoricidal activity. This approach can improve phagocytosis, boost antigen presentation, and stimulate the secretion of additional pro-inflammatory cytokines, thereby strengthening the anti-tumor immune response. These properties contribute to reshaping the tumor microenvironment to favor anti-tumor immunity, making interleukin-12 a promising target for cancer immunotherapy and a key factor in harnessing M1 macrophage functions.

Currently, no interleukin-12-based therapeutics have been approved for clinical application because of its dose-limiting toxicities by systemic administration (Haicheur, N., B. Escudier, et al. (2000). “Cytokines and soluble cytokine receptor induction after IL-12 administration in cancer patients.” Clin Exp Immunol 119(1): 28-37.). It is reported that a single intratumoral dose of mouse interleukin-12 mRNA induced tumor regression in multiple tumor models has been described (Hewitt, S. L., D. Bailey, et al. (2020). “Intratumoral IL12 mRNA Therapy Promotes TH1 Transformation of the Tumor Microenvironment.” Clin Cancer Res 26(23): 6284-6298.). Therefore, delivery of interleukin-12 into a confined tissue and tumor sites may be a feasible approach to avoid systemic toxicities.

CD47 is a transmembrane protein that functions as a “don't eat me” signal by interacting with signal regulatory protein alpha (SIRPα) on macrophages, thereby inhibiting phagocytosis (Logtenberg, M. E. W., F. A. Scheeren, et al. (2020). “The CD47-SIRPalpha Immune Checkpoint.” Immunity 52(5): 742-752.). Various solid tumors (for example, liver, breast, ovarian, bladder, colon, pancreatic, lung cancers) overexpress CD47 to evade immune surveillance, making it a promising tumor-associated antigen (TAA) for cancer therapy.

Targeting CD47 in solid tumor treatment has several advantages. First, blocking the CD47-SIRPα interaction restores macrophage-mediated phagocytosis, allowing the immune system to effectively eliminate tumor cells. Second, this strategy enhances antigen presentation by macrophages, promoting the activation of cytotoxic T cells and strengthening adaptive immunity. Third, CD47 blockade can remodel the tumor microenvironment by shifting macrophages from an immunosuppressive M2 phenotype to a pro-inflammatory M1 phenotype, further enhancing anti-tumor activity. Furthermore, CD47 is widely expressed across various solid tumors, making it a broad-spectrum therapeutic target. Accordingly, CD47 targeting strategies offers a powerful strategy for enhancing both innate and adaptive immune responses against solid tumors.

Based on the foregoing, the present disclosure designs an mRNA-LNP-based novel nucleic acid drug to promote the in vivo generation of M1-polarized CAR macrophages for cancer therapy. The advantages of the present invention are as follows.

Macrophages play a pivotal role in cancer immunity, including tumor infiltration, phagocytosis, antigen presentation, and modulation of adaptive immunity for tumor elimination.

Using interleukin-12 mRNA as composition can drives macrophage polarization toward an M1 pro-inflammatory phenotype, boosting their anti-tumor activity.

The chimeric antigen receptor-mRNA is translated into an anti-CD47 chimeric antigen receptor (CAR) protein, enabling macrophages to recognize tumor-associated antigens and enhance tumor phagocytosis, cytokine secretion, and antigen presentation.

A combination of CD47-targeted chimeric antigen receptor mRNA and interleukin-12 mRNA can eliminate the need for additional administration of immune checkpoint inhibitors in cancer therapy.

Both IL-12 and anti-CD47 CAR mRNAs are encapsulated within lipid nanoparticles, protecting the mRNA from degradation and ensuring efficient uptake by M2 macrophages.

Remodeling the tumor microenvironment: chimeric antigen receptor-macrophages infiltrate the tumor microenvironment, these M1-polarized chimeric antigen receptor-macrophages actively engage in tumor cell phagocytosis, antigen presentation, and inflammatory cytokine release, promoting a robust immune activation against solid tumors.

In vivo delivery of mRNA-LNP into macrophages eliminates the need for ex vivo cell engineering, reducing logistical and manufacturing complexity.

This novel mRNA-LNP therapy harnesses both innate and adaptive immunity, offering a highly scalable and effective treatment for solid tumors that are resistant to conventional immunotherapies.

Integrating the above description and referring to FIG. 1, it is understood that the present disclosure can at least provide the following contents, but it is not limited thereto.

The present disclosure may provide an mRNA composition for treating cancer.

The aforementioned cancer is not particularly limited, as long as its cancer cells can express CD47. In one embodiment, the aforementioned cancer may comprise a solid tumor, but it is not limited thereto. In another embodiment, examples of the aforementioned cancers may comprise, but are not limited to, liver cancer, lung cancer, pancreatic cancer, kidney cancer, spleen cancer, lymphoma, breast cancer, ovarian cancer, uterine cancer, prostate cancer, bladder cancer, colon cancer, rectal cancer, brain cancer and spinal cord cancer. In yet another embodiment, the aforementioned cancer may comprise carcinoma in situ, invasive cancer, or metastatic cancer, but it is not limited thereto. In one specific embodiment, the aforementioned cancer may comprise, but is not limited to, liver cancer. In another specific embodiment, the aforementioned cancer may comprise metastatic liver cancer, but it is not limited thereto.

The mRNA composition for treating cancer mentioned above may comprise, but is not limited to, an mRNA encoding a CD47-targeted chimeric antigen receptor (CAR) and an mRNA encoding interleukin 12 (IL-12).

The mRNA composition for treating cancer disclosed above can promote the in vivo generation of M1 polarized chimeric antigen receptor macrophages for cancer therapy. Moreover, the mRNA of a CD47-targeted chimeric antigen receptor (CAR) is translated into the CD47-targeted chimeric antigen receptor (CAR) protein in vivo, enabling macrophages to recognize tumor-associated antigens and enhance tumor phagocytosis, cytokine secretion and antigen presentation. Furthermore, interleukin-12 mRNA as a component can drive macrophage polarization toward the M1 pro-inflammatory phenotype and enhance its anti-tumor activity.

The CD47-targeted chimeric antigen receptor encoded by the CD47-targeted chimeric antigen receptor (CAR) mRNA mentioned above may comprise, but is not limited to, an extracellular domain, a transmembrane domain, and an intracellular domain. The transmembrane domain may be located between the extracellular domain and the intracellular domain and connect the extracellular domain to the intracellular domain.

In one embodiment, the extracellular domain of the CD47-targeted chimeric antigen receptor mentioned above may comprise, but is not limited to, a CD47 recognition domain. The CD47 recognition domain mentioned above may comprise, but is not limited to, any molecule that can bind to CD47, for example, an anti-CD47 antibody fragment, signal regulatory protein alpha (SIRPα) with enhanced binding ability to CD47, but it is not limited thereto. The anti-CD47 antibody fragment mentioned above may comprise, but is not limited to, a single domain antibody fragment (sdAb) that can specifically bind to CD47, a single-chain variable fragment (scFv) that can specifically bind to CD47, etc.

Furthermore, in another embodiment, the extracellular domain of the CD47-targeted chimeric antigen receptor may further include a hinge region in addition to the CD47 recognition domain, but it is not limited thereto, and the hinge region may be connected to the transmembrane domain. The hinge region may comprise, but is not limited to, a CD28 derived hinge region.

In one embodiment, the transmembrane domain of the CD47-targeted chimeric antigen receptor mentioned above may include, but is not limited to, a CD28 derived transmembrane domain.

In addition, the intracellular domain mentioned above may comprise a CD28 derived intracellular T-cell costimulatory domain and a CD3ζ derived intracellular T-cell signaling domain, but it is also not limited thereto.

In one embodiment, in the foregoing mRNA composition for treating cancer, the sequence of the foregoing mRNA encoding a CD47-targeted chimeric antigen receptor, from the 5′ end to the 3′ end, may comprise an mRNA sequence encoding a CD47 recognition domain, an mRNA sequence encoding a CD28 derived intracellular T-cell costimulatory domain and an mRNA sequence encoding a CD3ζ derived intracellular T-cell signaling domain, but it is not limited thereto.

In another embodiment, the sequence of the foregoing mRNA encoding a CD47-targeted chimeric antigen receptor may comprise, but is not limited to, a sequence having 75% or more sequence identity with SEQ ID NO: 7. In one specific embodiment of this embodiment, the sequence of the foregoing mRNA encoding a CD47-targeted chimeric antigen receptor, from the 5′ end to the 3′ end, may comprise, but is not limited to, a 5′ cap, the sequence of SEQ ID NO: 7, and a 3′ poly-A tail.

In yet another embodiment, the sequence of the foregoing mRNA encoding a CD47-targeted chimeric antigen receptor may comprise, but is not limited to, a sequence having 75% or more sequence identity with SEQ ID NO: 5. In one specific embodiment of this embodiment, the sequence of the foregoing mRNA encoding a CD47-targeted chimeric antigen receptor, from the 5′ end to the 3′ end, may comprise, but is not limited to, a 5′ cap, the sequence of SEQ ID NO: 5, and a 3′ poly-A tail.

Furthermore, the foregoing poly-A tail may comprise, but is not limited to, adenosine, and the number of nucleotides in the poly-A tail is not particularly limited. In one embodiment, the number of nucleotides in the poly-A tail may be about 50-250, such as 50, 75, 100, 125, 150, 175, 200, 225 and 250, but it is not limited thereto.

The term “having 75% or more sequence identity with” used in the present disclosure means that there is an identity of about 75%-100% between two sequences, such as about 75%, about 80%, about 85%, about 90%, about 95%, about 96%, about 97%, about 98%, about 99%, about 99.5%, about 99.8%, about 100%, but it is not limited thereto.

Moreover, with respect to the mRNA composition for treating cancer mentioned above, in one embodiment, the interleukin-12 mentioned above may comprise, but is not limited to, a single chain interleukin-12. The single chain interleukin-12 mentioned above may comprise a signal peptide, an interleukin-12 β subunit protein, and an interleukin-12 α subunit protein, but it is not limited thereto.

In the mRNA composition for treating cancer mentioned above, in one embodiment, the sequence of the mRNA encoding interleukin-12 mentioned above, from the 5′ end to the 3′ end, may comprise, but is not limited to, an mRNA sequence encoding the signal peptide mentioned above, an mRNA sequence encoding the interleukin-12 β subunit protein mentioned above, and an mRNA sequence encoding the interleukin-12 α subunit protein mentioned above.

In another embodiment, the sequence of the foregoing mRNA encoding interleukin-12 may comprise a sequence having 75% or more sequence identity with SEQ ID NO: 7, but it is not limited thereto. In one specific embodiment of this embodiment, the sequence of the foregoing mRNA encoding interleukin-12, from the 5′ end to the 3′ end, may comprise, but is not limited to, a 5′ cap, the sequence of SEQ ID NO: 8, and a 3′ poly-A tail.

In yet another embodiment, the sequence of the foregoing mRNA encoding interleukin-12 may comprise a sequence having 75% or more sequence identity with SEQ ID NO: 6, but it is not limited thereto. In one specific embodiment of this embodiment, the sequence of the foregoing mRNA encoding interleukin-12, from the 5′ end to the 3′ end, may comprise, but is not limited to, a 5′ cap, the sequence of SEQ ID NO: 6, and a 3′ poly-A tail.

In addition, the foregoing poly-A tail may comprise, but is not limited to, adenosine, and the number of nucleotides in the poly-A tail is not particularly limited. In one embodiment, the number of nucleotides in the poly-A tail may be about 50-250, such as 50, 75, 100, 125, 150, 175, 200, 225 and 250, but it is not limited thereto.

Furthermore, in the mRNA composition for treating cancer mentioned above, in one embodiment, the mRNA encoding a CD47-targeted chimeric antigen receptor mentioned above and the mRNA encoding interleukin-12 mentioned above may be located in two separate single strands. The weight ratio of the mRNA encoding a CD47-targeted chimeric antigen receptor mentioned above to the mRNA encoding interleukin-12 mentioned above in the composition may be about 1-10:1-10, such as 2-9:2-9, 3-8:3-8, 4-7:4-7, 5-6:5-6, 1:1, 1:2, 1:3, 1:4, 1:5, 1:6, 1:7, 1:8, 1:9, 2:1, 3:1, 4:1, 5:1, 6:1, 7:1, 8:1, 9:1, 10:1, 3:4, 4:3, 4:5, 5:4, 3:7, 7:3, 8:5, 5:8, 9:7, 7:9, but it is not limited thereto. In one specific embodiment, the weight ratio of the mRNA encoding a CD47-targeted chimeric antigen receptor mentioned above to the mRNA encoding interleukin-12 mentioned above in the composition may be about 1:1. In another specific embodiment, the weight ratio of the mRNA encoding a CD47-targeted chimeric antigen receptor mentioned above to the mRNA encoding interleukin-12 mentioned above in the composition may be about 7:3.

Moreover, in another embodiment, in the mRNA composition for treating cancer mentioned above, the mRNA encoding a CD47-targeted chimeric antigen receptor mentioned above and the mRNA encoding interleukin-12 mentioned above may be connected by a linker to form a single strand. Examples of the aforementioned linker may comprise, but are not limited to, an internal ribosome entry site (IRES), a 2A peptide. Examples of 2A peptides may comprise P2A, E2A, F2A, T2A, but they are not limited thereto.

Furthermore, the aforementioned mRNA composition for treating cancer disclosed above, in addition to the aforementioned mRNA encoding a CD47-targeted chimeric antigen receptor and the aforementioned mRNA encoding interleukin-12, may further comprise, but is not limited to, a lipid-based nanocarrier (LBC), an exosome carrier, a viral vector, etc., for encapsulating or carrying the aforementioned mRNA encoding a CD47-targeted chimeric antigen receptor and the aforementioned mRNA encoding interleukin-12.

Examples of the aforementioned lipid-based nanocarrier may comprise, a lipid nanoparticle (LNP), a liposome, a nanoemulsions (NE), a nanocapsule, a self-emulsifying drug delivery system (SEDDS), but they are not limited thereto. Examples of the aforementioned viral vector may comprise, but are not limited to, a Lentiviral vector, an adenoviral vector, but they are also not limited thereto.

A composition used to form the foregoing lipid nanoparticle may comprise, but is not limited to, an ionizable amino lipid, a phospholipid, a cholesterol and a PEGylated lipid.

The aforementioned ionizable amino lipid may comprise heptadecan-9-yl 8-((2-hydroxyethyl)(6-oxo-6-(undecyloxy)hexyl)amino)octanoate (SM-102), (6Z,9Z,28Z,31Z)-Heptatriaconta-6,9,28,31-tetraen-19-yl 4-(dimethylamino)butanoate (D-Lin-MC3-DMA), [(4-Hydroxybutyl)azanediyl]di(hexane-6,1-diyl) bis(2-hexyldecanoate) (ALC-0315) or any combination thereof, but are not limited thereto.

Examples of the aforementioned phospholipids may include, but are not limited to, 1,2-dioleoyl-sn-glycero-3-phosphoethanolamine (DOPE), dimyristoylphosphatidylcholine (DMPC), 1,2-distearoyl-sn-glycero-3-phosphocholine (DSPC), dimyristoylphosphatidylglycerol (DMPG), dipalmitoylphosphatidylcholine (DPPC), phosphatidylcholine (PC) and any combination thereof.

Example of the aforementioned PEGylated lipid may comprise, DMG-PEG, but it is not limited thereto.

In addition, the present disclosure may also provide an mRNA pharmaceutical preparation for treating cancer, which may comprise, but is not limited to, any mRNA composition for treating cancer of the present application mentioned above. Relevant description for the cancer can be referred to the preceding related paragraphs which describe the mRNA composition for treating cancer of the present disclosure, and thus is not repeated herein.

In addition, the present disclosure may further provide a lipid nanoparticle preparation for treating cancer.

The aforementioned lipid nanoparticle preparation for treating cancer of the present disclosure may comprise, but is not limited to, a lipid nanoparticle, an mRNA encoding a CD47-targeted chimeric antigen receptor and an mRNA encoding interleukin-12. The aforementioned lipid nanoparticle may be loaded with the mRNA encoding a CD47-targeted chimeric antigen receptor and the mRNA encoding interleukin-12. Relevant description for the cancer can be referred to the preceding related paragraphs which describe the mRNA composition for treating cancer of the present disclosure, and thus is not repeated herein.

The lipid nanoparticle preparation for treating cancer of the present disclosure mentioned above can promote the in vivo generation of M1 polarized chimeric antigen receptor macrophages for cancer therapy. The mRNA of a CD47-targeted chimeric antigen receptor is translated into a chimeric antigen receptor (CAR) protein targeting CD47 in vivo, enabling macrophages to recognize tumor-associated antigens and enhance tumor phagocytosis, cytokine secretion and antigen presentation. Meanwhile, interleukin-12 mRNA as a component can drive macrophage polarization toward the M1 pro-inflammatory phenotype and enhance its anti-tumor activity. Moreover, the mRNA encoding a CD47-targeted chimeric antigen receptor and the mRNA encoding interleukin-12 are both encapsulated in a lipid nanoparticle, protecting the mRNA from degradation and ensuring effective uptake by M2 macrophages.

Relevant description for the mRNA encoding a CD47-targeted chimeric antigen receptor and the mRNA encoding interleukin-12 can be referred to the preceding related paragraphs which describe the mRNA composition for treating cancer of the present disclosure, and thus is not repeated herein.

In one embodiment, the lipid nanoparticle preparation for treating cancer of the present disclosure mentioned above may be an injection dosage form or an infusion dosage form, etc., but it is not limited thereto. In one specific embodiment, the lipid nanoparticle preparation for treating cancer of the present disclosure mentioned above may be an intravenous injection dosage form, and its composition may be delivered to the liver via intravenous injection. Moreover, in another specific embodiment, the lipid nanoparticle preparation for treating cancer of the present disclosure mentioned above may be an intravenous injection dosage form, and its composition may be formulated with different components in a conventional manner to make it have the function of distribution in other organs and tissues to deliver the mRNA encoding a CD47-targeted chimeric antigen receptor and the mRNA encoding interleukin-12 into the organ, so as to achieve the purpose of the treatment of cancers in other organs. In yet another embodiment, the lipid nanoparticle preparation of the present disclosure mentioned above may also be an intravenous injection dosage form using lipid nanoparticles whose surfaces are modified with molecules that can specifically target macrophages, but it is not limited thereto.

Furthermore, according to the foregoing, the present disclosure may also provide a use of an mRNA encoding a CD47-targeted chimeric antigen receptor and an mRNA encoding interleukin-12 in the manufacture of a medicament for treating cancer. The foregoing medicament may be capable of polarizing a M2 macrophage into a M1 macrophage to secrete interleukin-12 and form a CD47-targeted chimeric antigen receptor-macrophage (CD47-targeted CAR-Macrophage, CD47-targeted CAR-M) in a subject in need thereof.

Furthermore, the foregoing subject may include, but is not limited to, a vertebrate. Moreover, the vertebrate mentioned above may include a fish, an amphibian, a reptile, a bird, or a mammal, etc., but it is not limited thereto. Examples of the mammal may include, but are not limited to, a human, an orangutan, a monkey, a horse, a donkey, a llama, a dog, a cat, a rabbit, a guinea pig, a rat, and a mouse. In one embodiment, the subject mentioned above may be a human.

Regarding the use of an mRNA encoding a CD47-targeted chimeric antigen receptor and an mRNA encoding interleukin-12 in the manufacture of a medicament for treating cancer mentioned above, relevant descriptions for the said cancer, mRNA encoding a CD47-targeted chimeric antigen receptor and mRNA encoding interleukin-12 can be referred to the preceding related paragraphs which describe the mRNA composition for treating cancer of the present disclosure, and thus they are not repeated herein.

In addition, the administration manner for the aforementioned medicament is not particularly limited, as long as the mRNA encoding a CD47-targeted chimeric antigen receptor and the mRNA encoding interleukin-12 can be translated in the subject to which the medicament is administered. For example, the administration manner for the aforementioned medicament may comprise, but is not limited to, intravenous administration, intraperitoneal administration, subcutaneous administration, intracutaneous administration, intramuscular administration, intraarticular administration, intraarterial administration, intrasynovial administration, intrasternal administration, intrathecal administration, intralesional administration, infusion techniques, electroporation or gene gun. In one embodiment, the administration manner for the aforementioned medicament is intravenous administration.

Similarly, the dosage form or delivery system for the aforementioned medicament is not particularly limited, as long as the mRNA encoding a CD47-targeted chimeric antigen receptor and the mRNA encoding interleukin-12 can be translated in the subject to which the medicament is administered. For example, the dosage form for the aforementioned medicament may include, but is not limited to, an injection dosage form. The aforementioned delivery system may include, but is not limited to, a lipid-based nanocarrier, an exosome carrier, a viral vector, etc. Relevant descriptions for the lipid-based nanocarrier, exosome carrier and viral vector described herein, can be referred to the preceding related paragraphs which describe the mRNA composition for treating cancer of the present disclosure, and thus they are not repeated herein. In another embodiment, the medicament may be a lipid nanoparticle preparation.

Based on the foregoing, it can be understood that the present disclosure may also provide a method for treating cancer.

The method for treating cancer may comprise, but is not limited to, administering a medicament manufactured from any use of an mRNA encoding a CD47-targeted chimeric antigen receptor and an mRNA encoding interleukin-12 in the manufacture of a medicament for treating cancer of the present disclosure mentioned above, any mRNA composition for treating cancer of the present disclosure mentioned above, any pharmaceutical preparation for treating cancer of the present disclosure mentioned above or any lipid nanoparticle preparation for treating cancer of the present disclosure mentioned above to a subject in need thereof.

Relevant descriptions for the cancer, mRNA encoding a CD47-targeted chimeric antigen receptor and mRNA encoding interleukin-12 described herein, can be referred to the preceding related paragraphs which describe the mRNA composition for treating cancer of the present disclosure, and thus they are not repeated herein.

The subject and administration manner to described herein, can be referred to the preceding related paragraphs which describe the use of an mRNA encoding a CD47-targeted chimeric antigen receptor and an mRNA encoding interleukin-12 in the manufacture of a medicament for treating cancer of the present disclosure mentioned above, and thus they are not repeated herein.

Furthermore, the present disclosure may also provide a use of an mRNA encoding a CD47-targeted chimeric antigen receptor in the manufacture of a medicament for treating cancer, wherein the medicament for treating cancer may be administered together with an mRNA encoding interleukin-12. Such administration can polarize a M2 macrophage into a M1 macrophage in a subject in need thereof, thereby secreting interleukin-12 and forming CD47-targeted chimeric antigen receptor-macrophage.

Relevant descriptions for the cancer, mRNA encoding a CD47-targeted chimeric antigen receptor and mRNA encoding interleukin-12 described herein, can be referred to the preceding related paragraphs which describe the mRNA composition for treating cancer of the present disclosure, and thus they are not repeated herein.

Moreover, the subject and administration manner described herein, can be referred to the preceding related paragraphs which describe the use of an mRNA encoding a CD47-targeted chimeric antigen receptor and an mRNA encoding interleukin-12 in the manufacture of a medicament for treating cancer of the present disclosure mentioned above, and thus they are also not repeated herein.

In addition, the respective administration manners for the aforementioned medicament and mRNA encoding interleukin-12 are not particularly limited, as long as the mRNA encoding a CD47-targeted chimeric antigen receptor and the mRNA encoding interleukin-12 can be translated in the subject to which the medicament and the mRNA encoding interleukin-12 are administered. For example, the respective administration manners for the aforementioned medicament and mRNA encoding interleukin-12 may comprise, but are not limited to, intravenous administration, intraperitoneal administration, subcutaneous administration, intracutaneous administration, intramuscular administration, intraarticular administration, intraarterial administration, intrasynovial administration, intrasternal administration, intrathecal administration, intralesional administration, infusion techniques, electroporation or gene gun. In one embodiment, the administration manner for the aforementioned medicament is intravenous administration.

Similarly, the respective dosage forms or delivery systems for the aforementioned medicament and mRNA encoding interleukin-12 are not particularly limited, as long as the mRNA encoding a CD47-targeted chimeric antigen receptor and the mRNA encoding interleukin-12 can be translated in the subject to which the medicament and the mRNA encoding interleukin-12 are administered. For example, the respective dosage forms for the aforementioned medicament and mRNA encoding interleukin-12 may include, but are not limited to, an injection dosage form. Moreover, for example, the respective delivery systems for the aforementioned medicament and mRNA encoding interleukin-12 may include, but are not limited to, a lipid-based nanocarrier, an exosome carrier, a viral vector. Relevant descriptions for the lipid-based nanocarrier, exosome carrier and viral vector described herein, can be referred to the preceding related paragraphs which describe the mRNA composition for treating cancer of the present disclosure, and thus they are not repeated herein. In another embodiment, the aforementioned medicament and mRNA encoding interleukin-12 may each be a lipid nanoparticle preparation.

Based on the foregoing, it can be also understood that the present disclosure may provide another method for treating cancer.

The method for treating cancer may comprise, but is not limited to, administering a medicament manufactured from any use of an mRNA encoding a CD47-targeted chimeric antigen receptor in the manufacture of a medicament for treating cancer of the present disclosure mentioned above and an mRNA encoding interleukin-12 to a subject in need thereof.

Relevant descriptions for the cancer, mRNA encoding a CD47-targeted chimeric antigen receptor and mRNA encoding interleukin-12 described herein, can be referred to the preceding related paragraphs which describe the mRNA composition for treating cancer of the present disclosure, and thus they are not repeated herein.

The respective administration manners and dosage forms or delivery systems for the aforementioned medicament and mRNA encoding interleukin-12 described herein can be referred to the preceding related paragraphs which describe the use of an mRNA encoding a CD47-targeted chimeric antigen receptor in the manufacture of a medicament for treating cancer of the present disclosure, and thus they are also not repeated herein.

In addition, the present disclosure may also provide a use of an mRNA encoding interleukin-12 in the manufacture of a medicament for treating cancer, wherein the medicament for treating cancer may be administered together with an mRNA encoding a CD47-targeted chimeric antigen receptor. Such administration can polarize a M2 macrophage into a M1 macrophage in a subject in need thereof, thereby secreting interleukin-12 and forming CD47-targeted chimeric antigen receptor-macrophage.

Relevant descriptions for the cancer, mRNA encoding interleukin-12 and mRNA encoding a CD47-targeted chimeric antigen receptor described herein, can be referred to the preceding related paragraphs which describe the mRNA composition for treating cancer of the present disclosure, and thus they are not repeated herein.

Furthermore, the subject described herein, can be referred to the preceding related paragraphs which describe the use of an mRNA encoding a CD47-targeted chimeric antigen receptor and an mRNA encoding interleukin-12 in the manufacture of a medicament for treating cancer of the present disclosure mentioned above, and thus it is also not repeated herein.

In addition, the respective administration manners for the aforementioned medicament and mRNA encoding a CD47-targeted chimeric antigen receptor are not particularly limited, as long as the mRNA encoding interleukin-12 and the mRNA encoding a CD47-targeted chimeric antigen receptor can be translated in the subject to which the medicament and the mRNA encoding a CD47-targeted chimeric antigen receptor are administered. For example, the respective administration manners for the aforementioned medicament and mRNA encoding a CD47-targeted chimeric antigen receptor may comprise, but are not limited to, intravenous administration, intraperitoneal administration, subcutaneous administration, intracutaneous administration, intramuscular administration, intraarticular administration, intraarterial administration, intrasynovial administration, intrasternal administration, intrathecal administration, intralesional administration, infusion techniques, electroporation or gene gun. In one embodiment, the administration manner for the aforementioned medicament is intravenous administration.

Similarly, the respective dosage forms or delivery systems for the aforementioned medicament and mRNA encoding a CD47-targeted chimeric antigen receptor are not particularly limited, as long as the mRNA encoding interleukin-12 and the mRNA encoding a CD47-targeted chimeric antigen receptor can be translated in the subject to which the medicament and the mRNA encoding a CD47-targeted chimeric antigen receptor are administered. For example, the respective dosage forms for the aforementioned medicament and mRNA encoding a CD47-targeted chimeric antigen receptor may include, but are not limited to, an injection dosage form. Moreover, for example, the respective delivery systems for the aforementioned medicament and mRNA encoding a CD47-targeted chimeric antigen receptor may include, but are not limited to, a lipid-based nanocarrier, an exosome carrier, a viral vector. Relevant descriptions for the lipid-based nanocarrier, exosome carrier and viral vector described herein, can be referred to the preceding related paragraphs which describe the mRNA composition for treating cancer of the present disclosure, and thus they are not repeated herein. In another embodiment, the aforementioned medicament and mRNA encoding a CD47-targeted chimeric antigen receptor may each be a lipid nanoparticle preparation.

Based on the foregoing, it can be also understood that the present disclosure may provide yet another method for treating cancer.

The method for treating cancer may comprise, but is not limited to, administering a medicament manufactured from any use of an mRNA encoding interleukin-12 in the manufacture of a medicament for treating cancer of the present disclosure mentioned above and an mRNA encoding a CD47-targeted chimeric antigen receptor to a subject in need thereof.

Relevant descriptions for the cancer, mRNA encoding interleukin-12 and mRNA encoding a CD47-targeted chimeric antigen receptor described herein, can be referred to the preceding related paragraphs which describe the mRNA composition for treating cancer of the present disclosure, and thus they are not repeated herein.

The respective administration manners and dosage forms or delivery systems for the aforementioned medicament and mRNA encoding a CD47-targeted chimeric antigen receptor described herein can be referred to the preceding related paragraphs which describe the use of an mRNA encoding interleukin-12 in the manufacture of a medicament for treating cancer of the present disclosure, and thus they are also not repeated herein.

### EXAMPLES

### Example 1

Construction of plasmids expressing CD47-targeted chimeric antigen receptor (CAR) and IL-12 mRNAs

A CD47-targeted chimeric antigen receptor was designed and synthesized, in which the chimeric antigen receptor contained a CD47-recognition domain comprised of a single domain antibody fragment (sdAb) derived from the murine single domain antibody A4 (Sockolosky, Dougan et al. 2016). The chimeric antigen receptor comprised a signal sequence from the mouse Ig kappa chain. Moreover, the chimeric antigen receptor also contained a combination of an intracellular T-cell costimulatory domain derived from the mouse CD28 and an intracellular T-cell signaling domain derived from the mouse CD3 zeta. More specifically, a cDNA, designated A4-28z, which encodes a chimeric antigen receptor protein comprising a mouse Ig kappa chain signal sequence, an A4 single domain antibody fragment (sdAb), a mouse CD28 transmembrane domain, an intracellular T-cell costimulatory domain of mouse CD28 and an intracellular T-cell signaling domain of mouse CD3ζ was constructed.

For the sake of conciseness, chimeric antigen receptor is referred to as CAR in the following.

The cDNA sequence coding for A4-28z is shown as the sequence of SEQ ID NO: 1 in the following. The coding region of A4-28z, from the 5′ to 3′ end, included a signal sequence of the mouse Ig kappa chain (boxed), the anti-mouse CD47 single domain antibody fragment (sdAb), A4 (shaded boldface), an EAAAK rigid linker (italics), a mouse CD28 hinge region (boxed boldface), a mouse CD28 transmembrane domain (lowercase), and a mouse CD28 intracellular T-cell costimulatory domain (shaded), and a mouse CD3ζ intracellular T-cell signaling domain (bold italics), followed by a TGA stop codon (boxed lowercase).

SEQ ID NO: 1

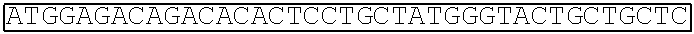

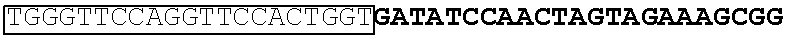

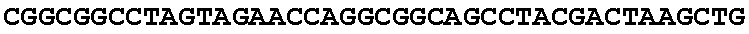

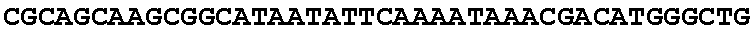

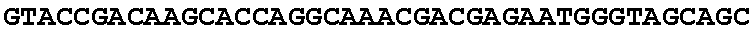

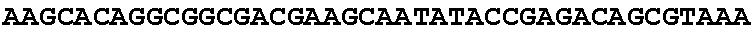

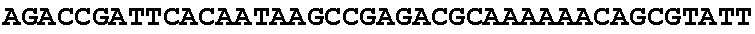

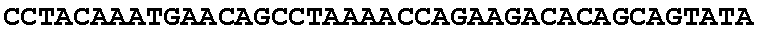

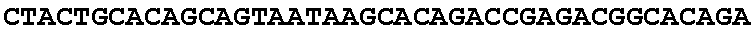

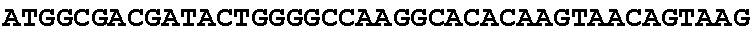

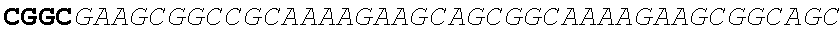

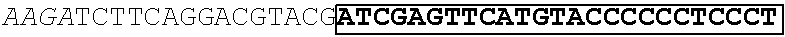

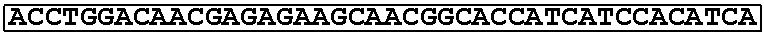

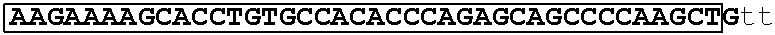

ctgggccctggtggtggtggccggcgtgctgttctgtttacggcctgctggtcacagtggccctgtgcg

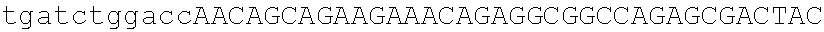

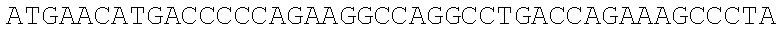

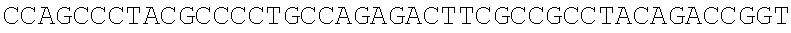

TAGAGCCAAGTTCAGCAGATCCGCCGAGACAGCCGCCAACCTGCA
GGATCCCAACCAGCTGTACAACGAGCTGAACCTGGGCAGACGGGA
GGAATACGACGTGCTGGAAAAGAAGAGAGCCAGGGACCCCGAGAT
GGGCGGCAAGCAGCAGAGAAGAAGAAACCCTCAGGAAGGCGTCTA
CAACGCCCTGCAGAAAGACAAGATGGCCGAGGCCTACAGCGAGAT
CGGCACCAAGGGCGAGAGAAGAAGGGGCAAGGGCCACGATGGCC
TGTACCAGGGCCTGTCCACCGCCACCAAGGACACCTACGACGCCC

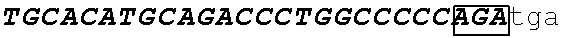

The cDNA mentioned above was codon-optimized and was subcloned into a modified expression vector of pUC19 (Cat. No. 50005, addgene), in which a T7 RNA polymerase-based in vitro transcription cassette was inserted at Kpn I and Hind III sites. Briefly, the T7 RNA polymerase-based in vitro transcription cassette consisted of a T7 promoter, a 5′ untranslated region (5′-UTR) (SEQ ID NO: 2, as shown in the following), a multiple cloning site, a 3′ untranslated region (3′-UTR) (SEQ ID NO: 3, as shown in the following), and a 189 nucleotide polyA tail sequence. The cDNA of A4-28z was subcloned at the multiple cloning sites Nco I and Xho I.

SEQ ID NO: 2
AGGAGACCCAAGCTGGCTAGGGTGGCGGGTTCTCTCTGAGTCTGT
GGGGACCAGAAGAATACTAGTGCCAC
SEQ ID NO: 3
CTCGAGCTGGTACTGCATGCACGCAATGCTAGATGCCCCTTTCCC
GTCCTGGGTACCCCGAGTCTCCCCCGACCTCGGGTCCCAGGTATG
CTCCCACCTCCACCTGCCCCACTCACCACCTCTGCTAGTTCCAGA
CACCTCCCAAGCACGCAGCAATGCAGCTCAAAACGCTTAGCCTAG
CCACACCCCCACGGGAAACAGCAGTGATTAACCTTTAGCAATAAA
CGAAAGTTTAACTAAGCTATACTAACCCCAGGGTTGGTCAATTTC
GTGCCAGCCACACCCCGGCCGC

The cDNA sequence coding for the single-chain mouse IL-12 is shown as the sequence of SEQ ID NO: 4 in the following. The coding region of the single-chain mouse IL-12, from the 5′ to 3′ end, included a signal sequence of human CD8 alpha (boxed), a mouse IL-12 beta subunit (p40) (boldface), a glycine linker (italics), a mouse IL-12 alpha subunit (p35) (italics, boldface, boxed), followed by a TGA stop codon (lowercase).

SEQ ID NO: 4

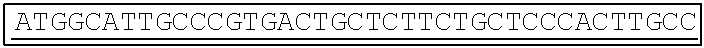

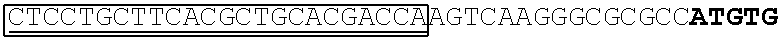

GGAGCTGGAGAAAGACGTTTATGTTGTAGAGGTGGACTGGAC
TCCCGATGCCCCTGGAGAAACAGTGAACCTCACCTGTGACAC
GCCTGAAGAAGATGACATCACCTGGACCTCAGACCAGAGACA
TGGAGTCATAGGCTCTGGAAAGACCCTGACCATCACTGTCAA
AGAGTTTCTAGATGCTGGCCAGTACACCTGCCACAAAGGAGG
CGAGACTCTGAGCCACTCACATCTGCTGCTCCACAAGAAGGA
AAATGGAATTTGGTCCACTGAAATTTTAAAAAATTTCAAAAAC
AAGACTTTCCTGAAGTGTGAAGCACCAAATTACTCCGGACGG
TTCACGTGCTCATGGCTGGTGCAAAGAAACATGGACTTGAAG
TTCAACATCAAGAGCAGTAGCAGTTCCCCTGACTCTCGGGCA
GTGACATGTGGAATGGCGTCTCTGTCTGCAGAGAAGGTCACA
CTGGACCAAAGGGACTATGAGAAGTATTCAGTGTCCTGCCAG
GAGGATGTCACCTGCCCAACTGCCGAGGAGACCCTGCCCATT
GAACTGGCGTTGGAAGCACGGCAGCAGAATAAATATGAGAAC
TACAGCACCAGCTTCTTCATCAGGGACATCATCAAACCAGACC
CGCCCAAGAACTTGCAGATGAAGCCTTTGAAGAACTCACAGG
TGGAGGTCAGCTGGGAGTACCCTGACTCCTGGAGCACTCCCC
ATTCCTACTTCTCCCTCAAGTTCTTTGTTCGAATCCAGCGCAA
GAAAGAAAAGATGAAGGAGACAGAGGAGGGGTGTAACCAGAA
AGGTGCGTTCCTCGTAGAGAAGACATCTACCGAAGTCCAATG
CAAAGGCGGGAATGTCTGCGTGCAAGCTCAGGATCGCTATTA
CAATTCCTCATGCAGCAAGTGGGCATGTGTTCCCTGCAGGGT
CCGATCCGGCGGCGGCGGGAGTGGCGGCGGGGGTTCTGGCGGAG

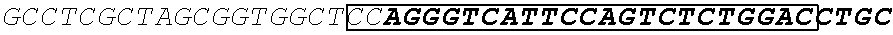

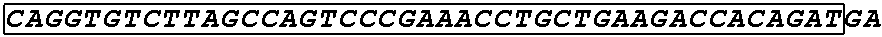

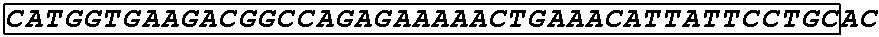

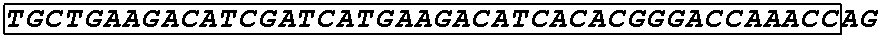

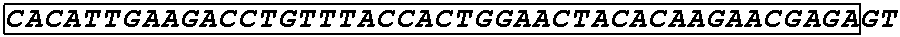

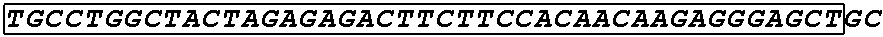

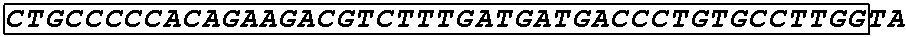

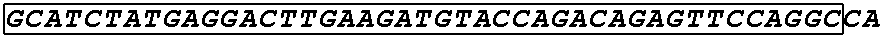

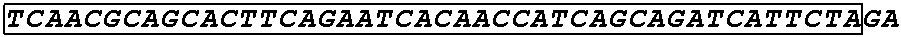

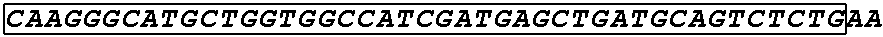

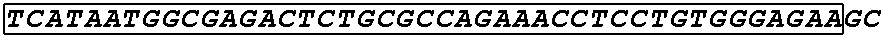

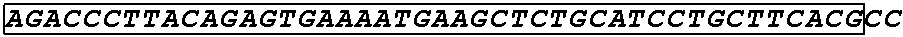

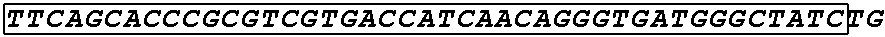

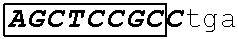

### Example 2

In Vitro Transcription and Purification of mRNA

First, all plasmids used for in vitro transcription reactions were linearized with Xba I restriction enzyme, followed by plasmid purification. RiboMAX™ Large Scale RNA Production Systems (cat no. P1300, Promega) was used for T7-RNA polymerase-based in vitro transcription reactions according to the manufacturer's instructions. Co-transcriptional capping approach was performed using CleanCap™ AG (^{m7}GpppAmG, cat no. N-7113-10, TriLink), and N1-methylpseudo-UTP (^{M1}Ψ, cat no. NU-890, Jena Bioscience GmbH) was used for nucleotide incorporation. The in vitro transcribed A4-28z and IL-12 mRNA sequences are shown in the following. Purified mRNA was assessed by spectrophotometry, gel electrophoresis, and BioAnalyzer (Agilent).

The content of the in vitro transcribed A4-28z mRNA from the 5′ to 3′ end is as follows:

5′ Cap: ^{m7}GpppAmG—the sequence of SEQ ID NO: 5-3′ polyA tail.

Moreover, the detailed sequence of SEQ ID NO: 5 is shown in the following:

A^{M1}ΨGGAGACAGACACAC^{M1}ΨCC^{M1}ΨGC^{M1}ΨA^{M1}ΨGGG^{M1}ΨAC^{M1}
ΨGC^{M1}ΨGC^{M1}ΨC^{M1}ΨGGG^{M1}Ψ^{M1}ΨCCAGG^{M1}Ψ^{M1}ΨCCAC^{M1}ΨGG^{M}
¹ΨGA^{M1}ΨA^{M1}ΨCCAAC^{M1}ΨAG^{M1}ΨAGAAAGCGGCGGCGGCC^{M1}ΨA
G^{M1}ΨAGAACCAGGCGGCAGCC^{M1}ΨACGAC^{M1}ΨAAGC^{M1}ΨGCGCAG
CAAGCGGCA^{M1}ΨAA^{M1}ΨA^{M1}Ψ^{M1}ΨPCAAAA^{M1}ΨAAACGACA^{M1}ΨG
GGC^{M1}ΨGG^{M1}ΨACCGACAAGCACCAGGCAAACGACGAGAA^{M1}ΨGG
G^{M1}ΨAGCAGCAAGCACAGGCGGCGACGAAGCAA^{M1}ΨA^{M1}ΨACCGA
GACAGCG^{M1}ΨAAAAGACCGA^{M1M1}CACAA^{M1}ΨAAGCCGAGACGCA
AAAAACAGCG^{M1}A^{M1}P^{M1}PCC^{M1}ΨACAAA^{M1}GAACAGCC^{M1}ΨAAA
ACCAGAAGACACAGCAG^{M1}A^{M1}ΨAC^{M1}PAC^{M1}ΨGCACAGCAG^{M1}Ψ
AA^{M1}ΨAAGCACAGACCGAGACGGCACAGAA^{M1}ΨGGCGACGA^{M1}ΨA
C^{M1}GGGGCCAAGGCACACAAG^{M1}ΨAACAG^{M1}ΨAAGCAGCGAAGCG
GCCGCAAAAGAAGCAGCGGCAAAAGAAGCGGCAGCAAGA^{M1}ΨC^{M1}
Ψ^{M1}Ψc^{M1}ΨACC^{M1}ΨGGACAACGAGAGAAGCAACGGCACCA^{M1}ΨCA
^{M1}ΨCCACA^{M1}ΨCAAAGAAAAGCACC^{M1}ΨG^{M1}ΨGCCACACCCAGAG
CAGCCCCAAGC^{M1}ΨG^{M1}Ψ^{M1}ΨC^{M1}ΨGGGCCC^{M1}ΨGG^{M1}ΨGG^{M1}ΨG
G^{M1}ΨGGCCGGCG^{M1}ΨGC^{M1}ΨG^{M1}Ψ^{M1}ΨC^{M1}ΨG^{M1}Ψ^{M1}ΨACGGCC^{M}
¹ΨGC^{M1}ΨGG^{M1}ΨCACAG^{M1}ΨGGCCC^{M1}Ψ^{M1}ΨGCG^{M1}ΨGA^{M1}ΨC^{M1}ΨG
GACCAACAGCAGAAGAAACAGAGGCGGCCAGAGCGAC^{M1}ΨACA
^{M1}ΨGAACA^{M1}ΨGACCCCCAGAAGGCCAGGCC^{M1}ΨGACCAGAAAGC
CC^{M1}ΨACCAGCCC^{M1}ΨACGCCCC^{M1}ΨCCAGAGAC^{M1}Ψ^{M1}ΨCGCCG
CC^{M1}ΨACAGACCGG^{M1}Ψ^{M1}ΨAGAGCCAAG^{M1}Ψ^{M1}ΨCAGCAGA^{M1}Ψ
CCGCCGAGACAGCCGCCAACC^{M1}ΨGCAGGA^{M1}ΨCCCAACCAGC^{M1}
ΨG^{M1}ΨACAACGAGC^{M1}ΨGAACC^{M1}ΨGGGCAGACGGGAGGAA^{M1}ΨA
CGACG^{M1}ΨGC^{M1}ΨGGAAAAGAAGAGAGCCAGGGACCCCGAGA^{M1}Ψ
GGGCGGCAAGCAGCAGAGAAGAAGAAACCC^{M1}ΨCAGGAAGGCG^{M1}
ΨC^{M1}ΨACAACGCCC^{M1}ΨCAGAAAGACAAGA^{M1}ΨGCCGAGGCC^{M1}ΨA
CAGCGAGA^{M1}ΨCGGCACCAAGGGCGAGAGAAGAAGGGGCAAGG
GCCACGA^{M1}ΨGGCC^{M1}ΨG^{M1}ΨACCAGGGCC^{M1}ΨG^{M1}ΨCCACCGCCA
CCAAGGACACC^{M1}ΨACGACGCCC^{M1}ΨGCACA^{M1}ΨGCAGACCC^{M1}Ψ
GGCCCCCAGA^{M1}ΨGA

The content of the in vitro transcribed IL-12 mRNA from the 5′ to 3′ end is as follows:

5′ Cap: ^{m7}GpppAmG—the sequence of SEQ ID NO: 6-3′ polyA tail.

Moreover, the detailed sequence of SEQ ID NO: 6 is shown in the following:

A^{M1}ΨGGCA^{M1}Ψ^{M1}ΨGCCCG^{M1}ΨGAC^{M1}ΨCG^{M1}ΨC^{M1}ΨC^{M1}Ψ^{M1}ΨC
^{M1}ΨGC^{M1}ΨCCCAC^{M1}Ψ^{M1}ΨGCCC^{M1}ΨCC^{M1}ΨGC^{M1}Ψ^{M1}ΨCACGC^{M1}Ψ
GCACGACCAAG^{M1}ΨCAAGGGCGCGCCA^{M1}ΨG^{M1}ΨGGGAGC^{M1}Ψ
GGAGAAAGACG^{M1}Ψ^{M1}Ψ^{M1}ΨA^{M1}ΨG^{M1}Ψ^{M1}ΨG^{M1}ΨAGAGG^{M1}ΨGGA
C^{M1}ΨGGAC^{M1}ΨCCCGA^{M1}ΨGCCCC^{M1}ΨGAGAAACAG^{M1}ΨGAACC
^{M1}ΨCACC^{M1}ΨG^{M1}ΨGACACGCC^{M1}ΨAAGAAGA^{M1}ΨACA^{M1}ΨCACC
^{M1}ΨGGACC^{M1}ΨCAGACCAGAGACA^{M1}ΨGGAG^{M1}ΨA^{M1}ΨAGGC^{M1}Ψ
C^{M1}ΨGGAAAGACCC^{M1}ΨGACCA^{M1}ΨCAC^{M1}ΨG^{M1}ΨCAAAGAG^{M1}Ψ
^{M1}Ψ^{M1}ΨC^{M1}ΨAGA^{M1}ΨGC^{M1}ΨGCCAG^{M1}ΨACACC^{M1}ΨGCCACAAA
GGAGGCGAGAC^{M1}ΨC^{M1}ΨGAGCCAC^{M1}ΨCACA^{M1}Ψ^{M1}ΨGC^{M1}ΨGC
^{M1}ΨCCACAAGAAGGAAAA^{M1}ΨGGAA^{M1}Ψ^{M1}Ψ^{M1}ΨGG^{M1}ΨCCAC^{M1}
ΨGAAA^{M1}Ψ^{M1}Ψ^{M1}Ψ^{M1}ΨAAAAAA^{M1}Ψ^{M1}Ψ^{M1}ΨCAAAAACAAGAC^{M}
¹Ψ^{M1}Ψ^{M1}ΨCC^{M1}ΨGAAG^{M1}ΨG^{M1}ΨGAAGCACCAAA^{M1}Ψ^{M1}ΨAC^{M1}
ΨCCGGACGG^{M1}Ψ^{M1}ΨCACG^{M1}ΨGC^{M1}ΨCA^{M1}ΨGGC^{M1}ΨGG^{M1}ΨGC
AAAGAAACA^{M1}ΨGGAC^{M1}Ψ^{M1}ΨGAAG^{M1}Ψ^{M1}ΨCAACA^{M1}ΨCAAGA
GCAG^{M1}ΨAGCAG^{M1}Ψ^{M1}ΨCCCC^{M1}ΨGAC^{M1}ΨC^{M1}ΨCGGGCAG^{M1}Ψ
GACA^{M1}ΨG^{M1}ΨGGAA^{M1}ΨGGCG^{M1}ΨC^{M1}ΨC^{M1}ΨG^{M1}ΨC^{M1}ΨGCAG
AGAAGG^{M1}ΨCACAC^{M1}ΨGGACCAAAGGGAC^{M1}ΨA^{M1}ΨGAGAAG
^{M1}Ψ^{M1}Ψ^{M1}ΨCAG^{M1}Ψ^{M1}ΨCC^{M1}ΨGCCAGGAGGA^{M1}ΨG^{M1}ΨCACC^{M1}Ψ
GCCCAAC^{M1}ΨGCCGAGGAGACCC^{M1}ΨGCCCA^{M1}Ψ^{M1}ΨGAAC^{M1}Ψ
GGCG^{M1}Ψ^{M1}ΨGGAAGCACGGCAGCAGAA^{M1}ΨAAA^{M1}ΨA^{M1}ΨGAG
AAC^{M1}ΨCAGCACCAGC^{M1}Ψ^{M1}ΨC^{M1}Ψ^{M1}ΨCA^{M1}ΨCAGGGACA^{M1}Ψ
CA^{M1}ΨAAACCAGACCCGCCCAAGAAC^{M1}Ψ^{M1}ΨCAGA^{M1}ΨGAAG
CC^{M1}Ψ^{M1}Ψ^{M1}ΨGAAGAAC^{M1}ΨCACAGG^{M1}ΨGGAGG^{M1}ΨCAGC^{M1}ΨG
GGAG^{M1}ΨACCC^{M1}ΨGAC^{M1}ΨCC^{M1}ΨGGAGCAC^{M1}ΨCCCCA^{M1}Ψ^{M1}Ψ
CC^{M1}ΨAC^{M1}Ψ^{M1}ΨC^{M1}ΨCCC^{M1}ΨCAG^{M1}Ψ^{M1}ΨC^{M1}Ψ^{M1}Ψ^{M1}ΨG^{M1}Ψ^{M1}Ψ
CGAA^{M1}ΨCCAGCGCAAGAAAGAAAAGA^{M1}ΨAAGGAGACAGAG
GAGGGG^{M1}ΨG^{M1}ΨAACCAGAAAGG^{M1}ΨCG^{M1}Ψ^{M1}ΨCC^{M1}ΨCG^{M1}ΨA
GAGAAGACA^{M1}ΨC^{M1}ΨACCGAAG^{M1}ΨCAA^{M1}ΨCAAAGGCGGGA
A^{M1}ΨG^{M1}ΨC^{M1}ΨGCG^{M1}ΨGCAAGC^{M1}ΨCAGGA^{M1}ΨCGC^{M1}ΨA^{M1}Ψ^{M1}
ΨACAA^{M1}Ψ^{M1}ΨCC^{M1}ΨCA^{M1}ΨGCAGCAAG^{M1}ΨGGGCA^{M1}ΨG^{M1}ΨG^{M1}
Ψ^{M1}ΨCCC^{M1}ΨGCAGGG^{M1}ΨCCGA^{M1}ΨCCGGCGGCGGCGGGAG^{M1}
ΨGCGGCGGGGG^{M1}Ψ^{M1}Ψc^{M1}ΨGGCGGAGGCC^{M1}ΨCGC^{M1}ΨAGCG
G^{M1}ΨGGC^{M1}ΨCCAGGG^{M1}ΨCA^{M1}Ψ^{M1}ΨCCAG^{M1}ΨC^{M1}Ψ^{M1}ΨGGAC
C^{M1}ΨGCCAGG^{M1}ΨG^{M1}ΨC^{M1}Ψ^{M1}ΨAGCCAG^{M1}ΨCCCGAAACC^{M1}Ψ
GC^{M1}ΨGAAGACCACAGA^{M1}ΨG^{ACAM1}ΨGG^{M1}ΨGAAGACACGGCCA
GAGAAAAAC^{M1}ΨGAAACA^{M1}Ψ^{M1}Ψ^{AM1}Ψ^{M1}ΨCC^{M1}ΨGCAC^{M1}ΨGC^{M1}
ΨGAAGACA^{M1}ΨCGA^{M1}ΨCA^{M1}ΨGAAGACA^{M1}ΨACACGGGACCA
AACCAGCACA^{M1}Ψ^{M1}ΨGAAGACC^{M1}ΨG^{M1}Ψ^{M1}Ψ^{M1}ΨACCAC^{M1}ΨG
GAAC^{M1}ΨACACAAGAACGAGAG^{M1}Ψ^{M1}ΨGCC^{M1}ΨGC^{M1}ΨAC^{M1}ΨA
GAGAGAC^{M1}Ψ^{M1}ΨC^{M1}Ψ^{M1}ΨCCACAACAAGAGGGAGC^{M1}ΨGCC^{M1}
ΨGCCCCCACAGAAGACG^{M1}Ψc^{M1}Ψ^{M1}Ψ^{M1}ΨGA^{M1}ΨGA^{M1}ΨGACCC
^{M1}Ψ^{M1}ΨGCC^{M1}Ψ^{M1}ΨGG^{M1}ΨAGCA^{M1}ΨC^{M1}ΨA^{M1}ΨGAGGAC^{M1}Ψ^{M1}Ψ
GAAGA^{M1}ΨG^{M1}ΨACCAGACAGAG^{M1}Ψ^{M1}ΨCCAGGCCA^{M1}ΨCAAC
GCAGCAC^{M1}Ψ^{M1}ΨCAGAA^{M1}ΨCACAACCA^{M1}ΨCAGCAGA^{M1}ΨCA
^{M1}Ψ1C^{M1}ΨAGACAAGGGCA^{M1}ΨGC^{M1}ΨGG^{M1}ΨGGCCA^{M1}ΨCGA^{M1}
ΨAGC^{M1}ΨGA^{M1}ΨGCAG^{M1}ΨC^{M1}ΨC^{M1}ΨGAA^{M1}ΨCA^{M1}ΨAA^{M1}ΨGGC
GAGAC^{M1}ΨC^{M1}ΨGCGCCAGAAACC^{M1}ΨCC^{M1}ΨMYGGGAGAAGC
AGACCC^{M1}Ψ^{M1}ΨACAGAG^{M1}ΨGAAAA^{M1}ΨGAAGC^{M1}ΨC^{M1}ΨGCA
^{M1}ΨCC^{M1}ΨGC^{M1}Ψ^{M1}ΨCACGCC^{M1}Ψ^{M1}ΨCAGCACCCGCG^{M1}ΨCG^{M1}
ΨGACCA^{M1}ΨCAACAGGG^{M1}ΨGA^{M1}ΨGGGC^{M1}Ψ^{M1}ΨC^{M1}ΨGAGC^{M1}
ΨCCGCC^{M1}ΨGA

### Example 3

Co-Transfection of IL-12 and CD47-Targeted CAR mRNAs can Induce M1 Polarization in M2 Mouse Macrophages

Since in solid tumor microenvironment, the infiltrating macrophages (including tumor-associated macrophages) are in tumor suppressive M2-like cell types (Noy and Pollard 2014), whether co-transfection of IL-12/A4-28z CAR mRNA into a M2-type macrophage can induce the reprogramming of M2-like macrophages to the M1 phenotype was determined in this experiment.

Mouse immortalized bone marrow-derived macrophages (iBMDMs) were treated with 40 ng/mL IL-4 for 48 hours to induce cell differentiation into M2-type macrophages. Afterwards, these M2 macrophages were co-transfected with an mRNA mixture of 1 g each of IL-12 mRNA and A4-28z CAR mRNA (IL-12/A4-28z mRNAs) by Lipofectamine MessengerMAX Reagent (cat no. LMRNA015, Thermo Fisher Scientific Inc.).

24 hours after transfection, cells were lysed and the gene expression profiles were analyzed via a reverse transcription quantitative polymerase chain reaction (RT-qPCR). The expression levels of the immunosuppressive M2 macrophage genes (Arg1, CD206, Fizz1, and Ym1) and M1 pro-inflammatory genes (IL-12p40, IL-1B, and iNOS) were quantitated to determine whether IL-12/A4-28z mRNAs co-transfection could induce M1 macrophage polarization, that is, converting M2 macrophages into an activated, tumor-suppressive M1 phenotype.

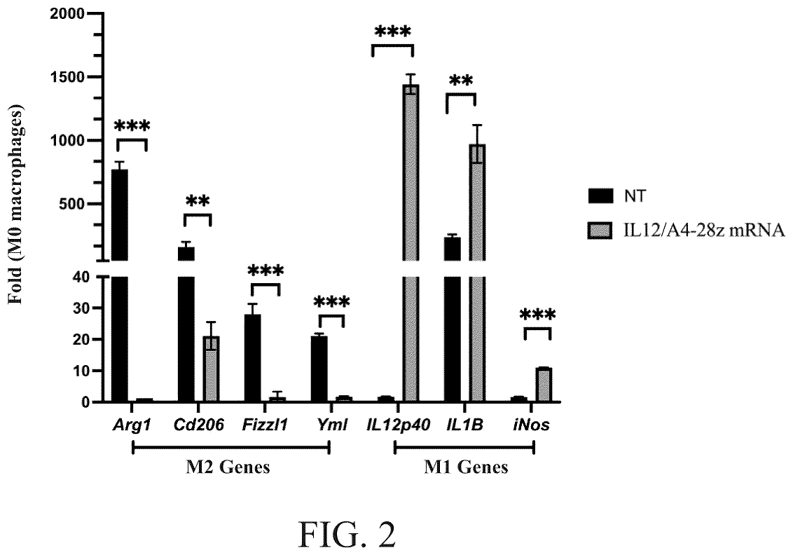

The results are shown in FIG. 2.

FIG. 2 shows that M2-type immortalized bone marrow-derived macrophages co-transfected with IL-12/A4-28z mRNAs can significantly suppress the expression of M2 macrophage-associated genes (M2 markers) and can promote the expression of M1 macrophage-associated genes (M1 markers). These findings demonstrate that IL-12/A4-28z mRNAs can effectively reprogram of M2-like macrophages to the M1 phenotype.

### Example 4

Co-Transfection of IL-12 and CD47-Targeted CAR mRNAs into Macrophages can Potentiate Macrophage-Mediated Phagocytosis of CD47+ Tumor Cells

In order to evaluate whether the expression of CD47-targeted CAR, A4-CD28z, can effectively block the CD47-SIRPα interaction and restore macrophage-mediated phagocytosis of CD47⁺ tumor cells, mouse IL-12 mRNA with or without the co-transfection of A4-CD28z mRNA was transfected into mouse immortalized bone marrow-derived macrophages, followed by co-culturing the above-mentioned mRNA-transfected mouse immortalized bone marrow-derived macrophages as effector cells (E) with a mouse CD47⁺ hepatocellular carcinoma cell line NHRI-8-B4 as target cells (T) (Cheng, Y. H., Y. C. Ko, et al. (2022). “Novel Paired Cell Lines for the Study of Lipid Metabolism and Cancer Stemness of Hepatocellular Carcinoma.” Front Cell Dev Biol 10: 821224) (kindly provided by Dr. Huang) to assess the tumor phagocytosis potency.

Macrophages were labeled with CMFDA (Green) fluorescent dyes, whereas NHRI-8-B4 cells were labeled with CMTPX (Red) fluorescent dyes and then they were co-cultured at an E:T ratio of 1:1 for 4 hours. Flow cytometry analysis was used to evaluate phagocytosis by detecting green-labeled macrophages that had engulfed tumor cells, as indicated by the presence of violet laser to felicitate red fluorescence signals (V610 nm). This allowed to assess the potency of CAR-mediated specific phagocytosis of tumor cells.

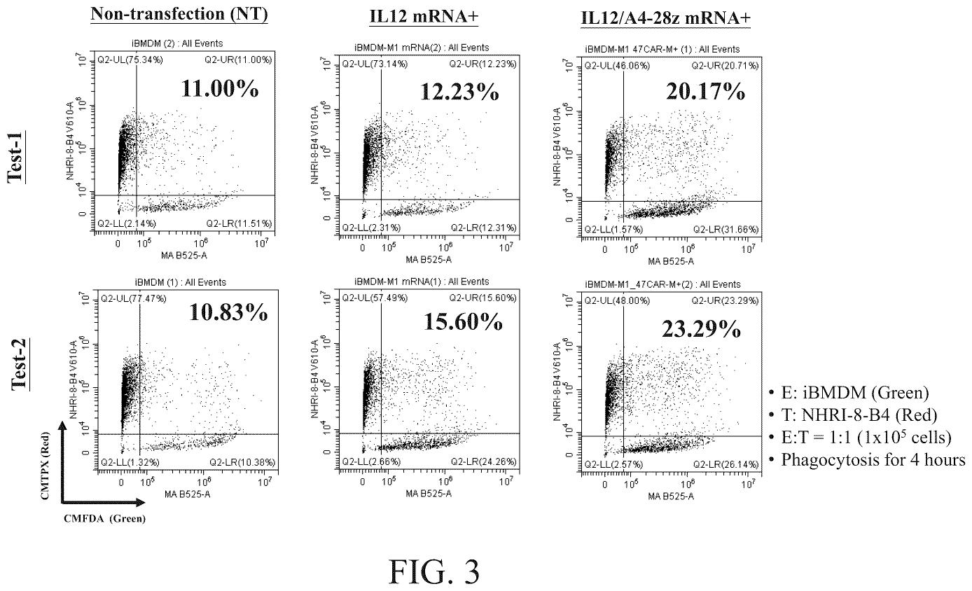

The results are shown in FIG. 3.

The non-transfected immortalized bone marrow-derived macrophages (indicated as NT (non-transfected)), which exhibited a spontaneous tumor cell phagocytosis potency of 10.83%˜11.00%, while immortalized bone marrow-derived macrophages expressing only IL-12 mRNA showed a slight increase in phagocytic activity (12.23%˜15.60%), possibly due to the “don't eat me” signaling event of CD47-SIRPα cellular interaction.

In contrast, immortalized bone marrow-derived macrophages expressing both IL-12 and A4-28z chimeric antigen receptor exhibited a significantly stronger tumor cell phagocytosis potency (20.17%˜23.29%).

These results indicate that using IL-12 mRNA can enhance macrophage-mediated tumor phagocytosis through M1 macrophage polarization, while introducing CD47-targeted CAR mRNA can effectively block the CD47-SIRPα interaction and restore macrophage-mediated phagocytosis of CD47⁺ tumor cells.

Therefore, designing an IL-12/CD47-targeted CAR mRNA-lipid nanoparticle (LNP) should be a feasible approach for solid tumor therapies.

### Example 5

Formulation of mRNA-Loaded Lipid Nanoparticle (LNP)

Sodium acetate buffer (10 mM, pH 4.0) and mRNAs were used to prepare the aqueous phase and Dlin-MC3-DMA (4-(dimethylamino)-, (10Z,13Z)-1-(9Z,12Z)-9,12-octadecadien-1-yl-10,13-nonadecadien-1-yl ester); IUPAC nomenclature, (6Z,9Z,28Z,31Z)-Heptatriaconta-6,9,28,31-tetraen-19-yl 4-(dimethylamino)butanoate; cat no. 555308, MedKoo Biosciences, Inc.), DOPE (1,2-dioleoyl-sn-glycero-3-phosphoethanolamine, cat no. 850725P, Merck KGaA), cholesterol (cat no. c8667, Merck KGaA), and DMG-PEG-2000 (cat no. 880151, Avanti Research) were mixed at molar ratios of 50:10:38.5:1.5 to prepare the ethanol phase.

The N/P ratio of Dlin-MC3-DMA to mRNA was set to be 6. In order to synthesize mRNA-LNPs, the ethanol phase containing lipids and cholesterol was mixed with the aqueous phase containing mRNA by alcohol dilution method. Briefly, while stirring the aqueous solution containing mRNA, the lipid ethanol solution was added by dropwise addition to form an mRNA-lipid nanoparticle solution.

The formed mRNA-lipid nanoparticle solution was then dialyzed against a 12-14 kDa MWCO Pur-A-Lyzer Maxi Dialysis kit (PURX12050, Sigma-Aldrich) at 4° C. overnight, and the mRNA-LNP was then sterilized with a 0.22 m filter. The formed mRNA-lipid nanoparticles were characterized using a Zetasizer Nano ZS90 (Malvern Instruments, Malvern, United Kingdom), measuring the average particle size, zeta potential and polydispersity index (PDI).

For the sake of conciseness, “lipid nanoparticle” is referred to as “LNP” in the following.

### Example 6

In Vivo Organ and Cellular Distribution of Cre mRNA-LNPs

The biodistribution of mRNA-LNP in mice was evaluated according to the published journal by Kauffman et. al. (Kauffman, K. J., M. A. Oberli, et al. (2018). “Rapid, Single-Cell Analysis and Discovery of Vectored mRNA Transfection In Vivo with a loxP-Flanked tdTomato Reporter Mouse.” Mol Ther Nucleic Acids 10: 55-63. Kauffman, Oberli et al. 2018).

Briefly, female Ai14 mice (B6.Cg-Gt(ROSA)26Sor^{tm14(AG-tdTomato)Hze}/J purchase from the Jackson Laboratory) were injected with 8.7 g/day of Cre mRNA-LNP (prepared in Example 5) intravenously for 2 consecutive days. 3 days after receiving the second dose, mice were sacrificed and major organs (heart, lung, liver, kidney, and spleen) were imaged using an in vivo imaging system (Caliper Life Sciences) to track the biodistribution of Cre mRNA, in which the tdTomato fluorescence was expressed following Cre-mediated recombination.

In order to track the cellular distribution of Cre-mRNA lipid nanoparticles (LNP) within specific cell populations in mouse liver tissue, the liver was dissociated into single-cell suspensions after administering the mRNA-LNPs. This was done using a semi-automated tissue processing device, the gentleMACS™ Dissociator (130-093-235, Miltenyi Biotec), in combination with the Liver Perfusion Kit (130-128-030, Miltenyi Biotec). The resulting cell suspension was subjected to low-speed centrifugation (50×g for 2 minutes), which produced two fractions: a pellet enriched with hepatocytes and a supernatant containing Kupffer cells (KCs) and liver sinusoidal endothelial cells (LSECs).

For hepatocyte isolation, the pellet was resuspended in RPMI-1640 medium and mixed 1:1 with Percoll, then centrifuged at 200×g for 10 minutes. The upper layer was discarded, and the viable hepatocytes from the lower layer were collected for subsequent flow cytometry antibody staining.

In order to isolate Kupffer cells and liver sinusoidal endothelial cells, the supernatant was centrifuged at 650×g for 10 minutes. The resulting pellet was resuspended in 17.6% Optiprep. Next, an 8.2% Optiprep solution was gently layered on top, followed by Hank's Balanced Salt Solution (HBSS). After that the gradient was centrifuged at 1400×g for 30 minutes, and the cells at the middle interface, enriched with Kupffer cells and liver sinusoidal endothelial cells, were collected for flow cytometry antibody staining.

Immunostaining and flow cytometry analyses were performed separately on the two cell populations (Kupffer cells and liver sinusoidal endothelial cells). Hepatocytes were initially stained with LIVE/DEAD™ Fixable Aqua (L34957, Invitrogen) to assess cell viability, followed by staining with an anti-albumin antibody (sc-271605, Santa Cruz) to identify the hepatocytes specifically.

Similarly, the Kupffer cells/liver sinusoidal endothelial cells fraction was stained with LIVE/DEAD™ Fixable Aqua (L34957, Invitrogen) to determine cell viability, followed by simultaneous immunostaining using the macrophage marker antibody anti-mouse F4/80 (123149, BioLegend) and the LSEC marker antibody anti-mouse CD31 (102435, BioLegend). Flow cytometry analyses were then conducted to quantify the cellular distribution of Cre-mRNA-LNP.

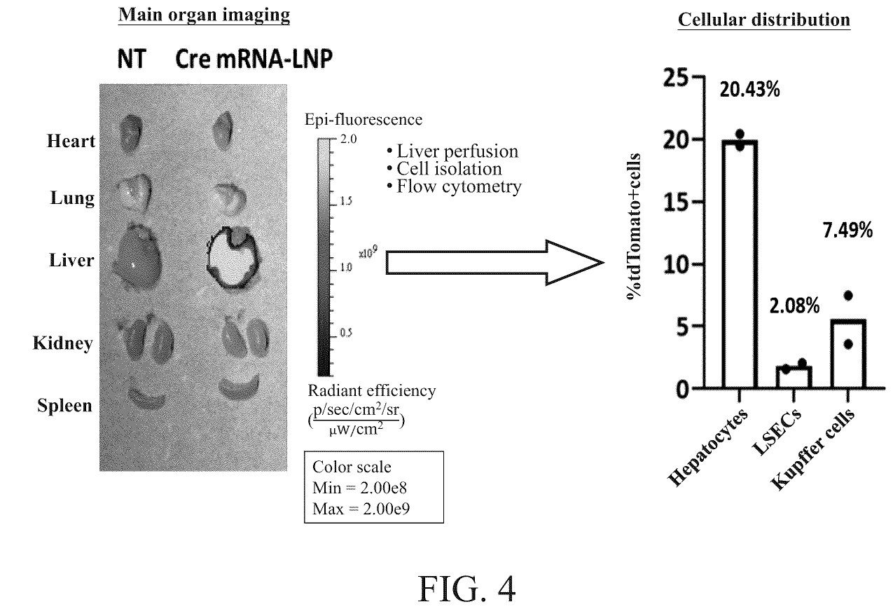

The results are shown in FIG. 4.

Following intravenous injection of Cre mRNA-LNP for 2 consecutive days, tdTomato fluorescent protein was only detected in the liver (FIG. 4, left panel). In order to identify which cell types were transfected and the transfection efficiency, flow cytometry was used to analyze the hepatocytes, Kupffer cells, and liver sinusoidal endothelial cells by staining with specific markers.

As shown in FIG. 4, right panel, it was found that hepatocytes were the major target cells for Cre mRNA-LNP delivery (20.43% in the gated hepatocyte population). Around 7.49% of the Kupper cells (liver-resident macrophages) were transfected by Cre mRNA-LNP and successfully expressed the tdTomato fluorescent protein.

These results indicated that mRNA formulated with the lipid nanoparticles in Example 3 can be specifically delivered to liver through intravenous injection. Furthermore, the results of cellular distribution imply that both hepatocytes and Kupper cells are capable of expressing the transfected mRNA payloads, including IL-12 and CARs.

### Example 7

IL-12/CD47-Targeted CAR mRNA-LNP Treatment Inhibits the Growth of Mouse Liver Tumor

In order to evaluate the tumor inhibition efficacy of IL-12/A4-28z CAR mRNA-LNP in inhibiting mouse liver tumor growth, a syngeneic C57BL/6J mouse spleen-liver cancer metastasis model was established by following the published protocol by Soares et al (Soares, K. C., K. Foley, et al. (2014). “A preclinical murine model of hepatic metastases.” J Vis Exp(91): 51677.).

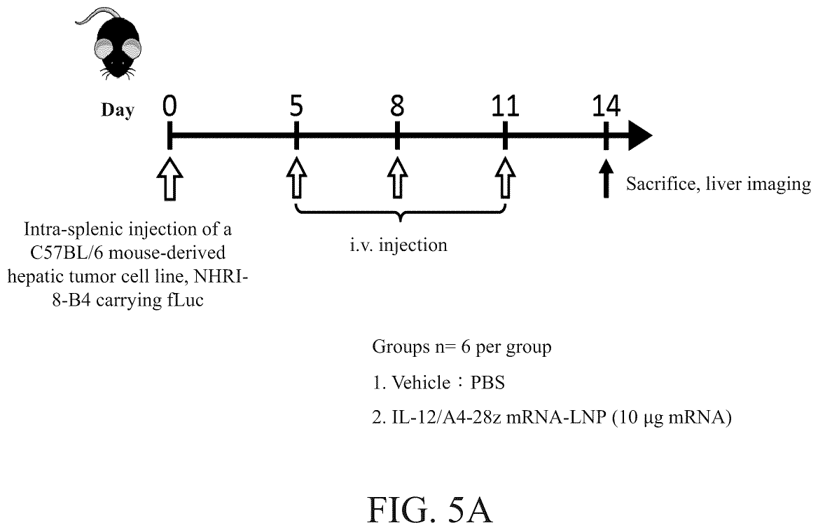

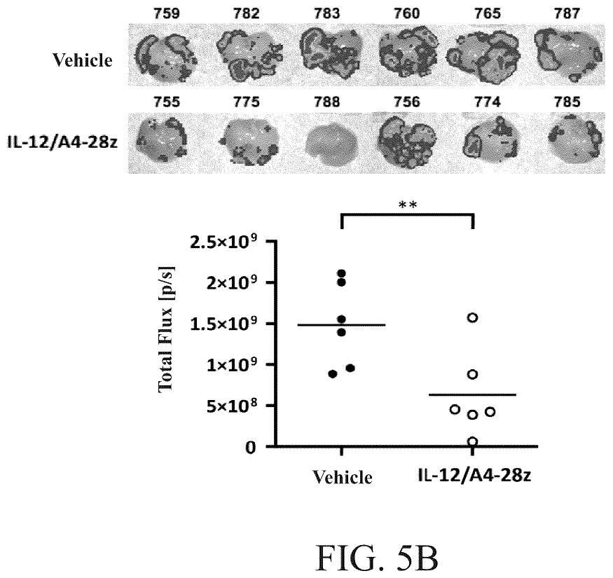

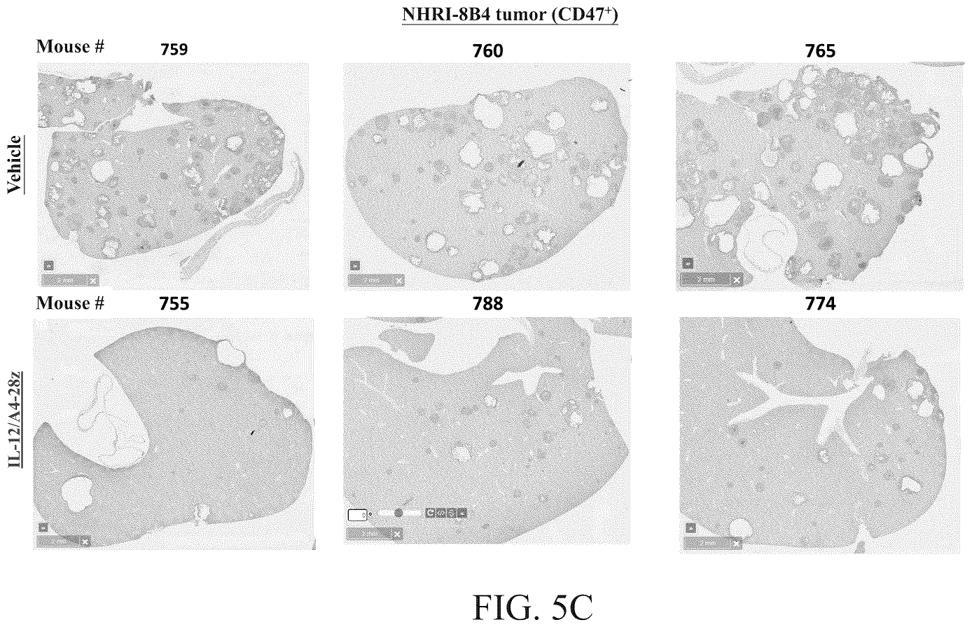

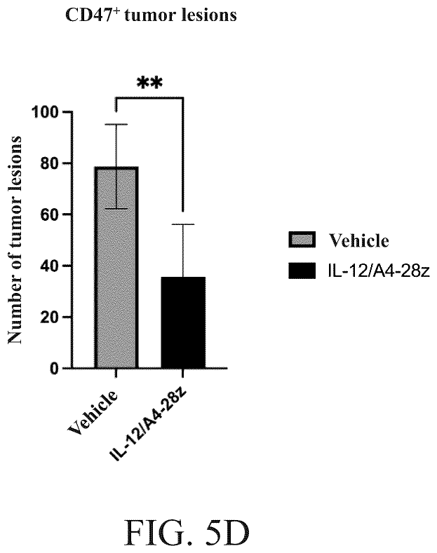

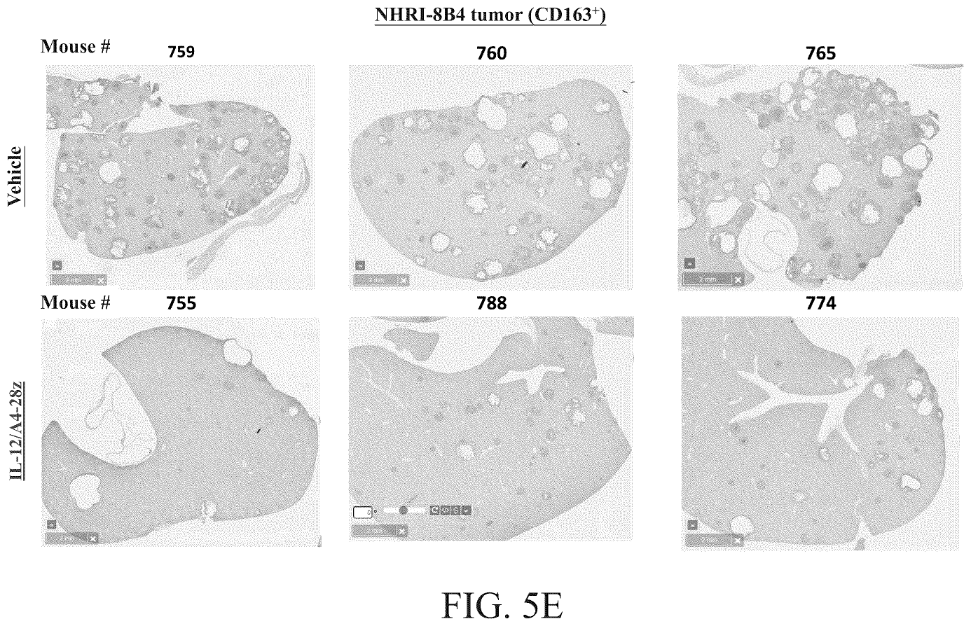

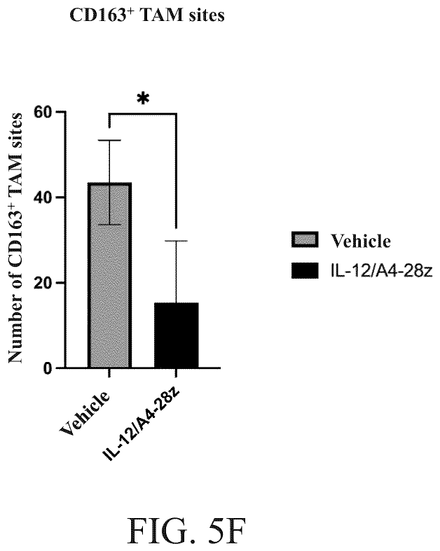

The experimental design is presented in FIG. 5A. In brief, 6-8-week-old female C57BL/6J mice were surgically implanted with C57BL/6J-derived mouse liver tumor cells, NRI-8-B4 (1×10⁵ cells per mouse), which carries firefly luciferase gene into the spleens. Tumor progression was monitored using in vivo imaging system (IVIS) imaging to detect luminescence signals. Once splenic tumor formation was confirmed (on Day 5 post-inoculation), mice were screened based on splenic bioluminescence intensities, and a total of 12 mice were selected and divided into 2 groups for study. Systemic intravenous administration was used for lipid nanoparticle delivery.

Each mouse in the experimental group received a formulated lipid nanoparticle carrying pre-mixed IL-12 mRNA (3 μg) and A4-28z mRNA (7 μg) payload (i.e., 10 μg mRNA/dose), while the control group (Vehicle) received phosphate buffer saline (PBS). Injections were administered every three days, with a total of three doses. 3 days (Day 14) after the final dose (Day 11), mice were euthanized and their livers were collected for assessments, including gross examination of hepatic tumor metastasis levels and histopathological immunostaining to evaluate the tumor lesions and tumor-associated macrophages (TAMs).

The results are shown in FIGS. 5B to 5F.

Compared to the vehicle control group, mice treated with IL-12/A4-28z CAR mRNA-LNP exhibited significantly lower numbers and sizes of NHRI-8-B4 tumor cell nodules in the liver, and one mouse was found to be free of tumor metastasis. Quantitative analysis of in vivo imaging system imaging data further confirmed that the bioluminescence signal intensity of liver tumors in the IL-12/A4-28z CAR mRNA-treated group was significantly lower than that in the vehicle group (FIG. 5B).

In addition to confirming that IL-12/A4-28z CAR mRNA-LNP inhibits liver tumor growth in the mouse liver cancer model, the changes in macrophage polarization within the tumor microenvironment following the treatment were also evaluated. In order to validate that IL-12/A4-28z CAR mRNA-LNP can effectively inhibit tumor growth in a mouse liver cancer model and to evaluate the changes in macrophage populations within the tumor microenvironment (TME) after CAR-M therapy, immunohistochemical (IHC) staining on pathological tissue sections was performed.

Specifically, tumor markers (CD47), M2 macrophage markers (CD206), tumor-associated M2 macrophage markers (CD163), and M1 macrophage markers (CD80⁺) were analyzed using immunohistochemistry. Image analysis software (ImageJ) was utilized to quantify and compare the number of CD47⁺ tumor lesions and CD163⁺ tumor-associated M2 macrophage infiltration areas (TAM-M2 sites).

The results showed that in mice treated with IL-12/A4-28z CAR mRNA-LNP, the number of CD47⁺ tumor lesions in liver tissue was significantly lower than that in the vehicle control group (FIG. 5B, *: p<0.05, n=3).

Similarly, immunohistochemical staining for M2 macrophage markers (CD206⁺) and tumor-associated M2 macrophage markers (CD163⁺) indicated that the expression levels of these two types of M2 macrophages were also lower in the IL12/A4-28z mRNA-LNP-treated group compared to the vehicle control group. Notably, the number of CD163⁺ tumor-associated macrophage infiltration areas (TAM-M2 sites) in liver tissues was significantly reduced in the IL-12/A4-28z CAR mRNA-LNP-treated group compared to the vehicle control group (FIGS. 5C to 5F, *: p<0.05, n=3).

These findings indicate that the treatment effectively inhibited the transformation of CD163⁺ tumor-associated macrophages (TAM-M2) within the liver tumor microenvironment. The results indicated that the IL-12/A4-28z CAR mRNAs promote the formation of M1 inflammatory macrophages with anti-tumor properties within the liver tumor microenvironment.

In conclusion, the results demonstrate that intravenous administration of IL-12/A4-28z CAR mRNA-LNP effectively inhibits liver tumor growth in the established mouse hepatocellular carcinoma model.

### Example 8

IL-12/CD47-Targeted CAR mRNA-LNP Treatment Effectively Inhibits Disseminated Tumor Cell Growth in Liver

In order to further investigate IL-12/A4-28z CAR mRNA-LNP mediated tumor inhibition in solid tumors, a syngeneic hepatic metastasis model by intravenous injection of A20 lymphoma cells (TIB-208, ATCC) was developed, in which A20 cells can spread, settle, and grow in the liver in a short period of time.

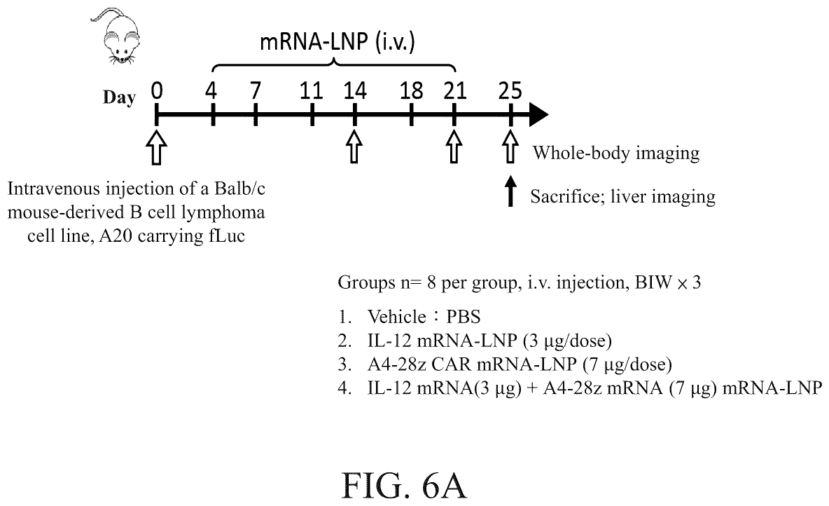

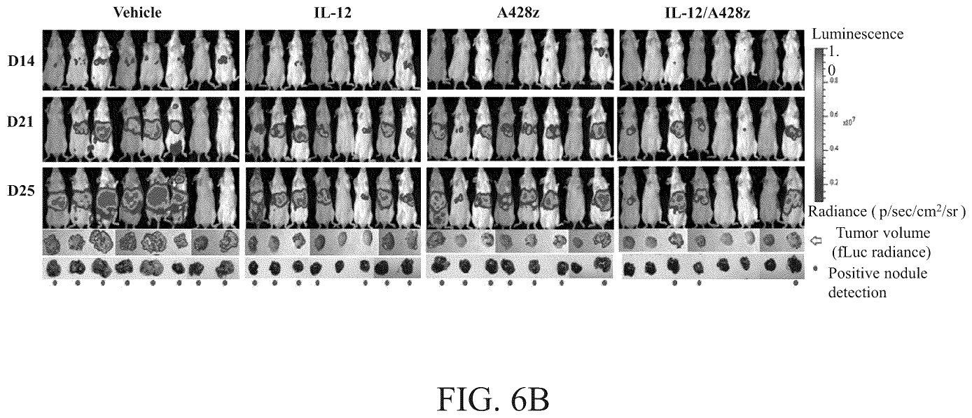

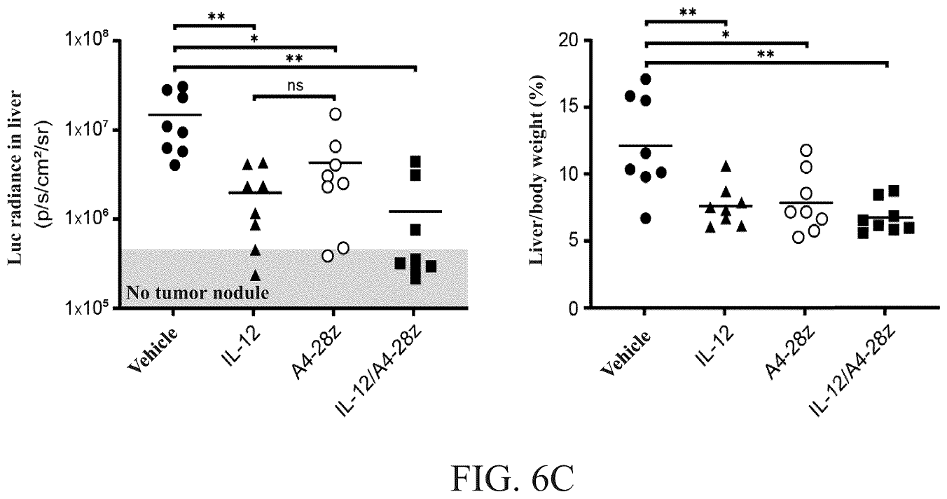

The experimental design is presented in FIG. 6A. In brief, on Day 0, A20-fLuc cells were administered intravenously into 6-8-week-old female BALB/c mice (2×10⁵ cells per mouse) to establish liver metastases. On Day 4, mice were divided into four experimental groups (n=8 per group) and the dosing regimen is as follows: (1) Group 1: PBS (negative control); (2) Group 2: IL-12 mRNA-LNP, 3 μg/dose; (3) Group 3: A4-28z mRNA-LNP, 7 μg/dose; (4) Group 4: LNP carrying pre-mixed IL-12 mRNA (3 μg) and A4-28z mRNA (7 μg) payload (i.e. 10 μg mRNA/dose).

All treatments were administered by intravenous injection twice weekly (BIW) for 3 weeks. Tumor progression and metastasis to the liver were assessed by in vivo imaging system bioluminescence imaging on Days 14, 21, and 25. On Day 25, mice were euthanized, and a gross examination of the liver was performed to quantify liver metastasis and tumor burden across experimental groups.

The results are shown in FIGS. 6B and 6C.

On Day 21 (D21), mice treated with IL-12/A4-28z mRNA-LNP exhibited significant inhibition of tumor growth in liver compared to the control groups. By Day 25 (D25), tumor suppression was even more pronounced, with 5 mice achieving completely free of tumor nodules. In order to further quantify liver metastasis, we measured bioluminescence intensity in excised liver tissues and calculated the liver-to-body weight ratio (FIG. 6C). Both analyses yielded consistent results, showing that neither IL-12 mRNA-LNP nor A4-28z mRNA-LNP alone can effectively inhibited liver tumor metastasis.

In contrast, only the combination of IL-12 and CD47-targeted CAR (IL-12/A4-28z) mRNA-LNP can successfully prevented tumor metastasis to the liver. These findings indicate that CD47-targeted CAR-expressing macrophages must be effectively polarized into M1-type macrophages to exert a robust tumor phagocytosis within the tumor microenvironment. In addition, this process facilitates cytotoxic T cells to eliminate tumor cells.

### Example 9

IL-12/CD47-Targeted CAR mRNA-LNP Treatment, without the Need of Immune Checkpoint Blockade, can Effectively Inhibit Disseminated Tumor Cell Growth in Liver

In order to further investigate whether combination of an immune checkpoint blockade, anti-PD-1 monoclonal antibody, with IL-12/A4-28z CAR mRNA-LNP can have any add-on effect on A20 tumor cell spreading and growth in liver, mice were divided in to the following four experimental groups (n=8 per group) for treatment and the dosing regimen is as follows: (1) Group 1: PBS (negative control); (2) Group 2: anti-mouse PD-1 monoclonal antibody (clone RMP1-14), 5 mg/kg body weight; (3) Group 3: LNP carrying pre-mixed IL-12 mRNA (3 μg) and A4-28z mRNA (7 μg) payload (i.e. 10 μg mRNA/dose); (4) Group 4: anti-mouse PD-1 monoclonal antibody (clone RMP1-14), 5 mg/kg body weight, and LNP carrying pre-mixed IL-12 mRNA (3 μg) and A4-28z mRNA (7 μg) payload (i.e. 10 μg mRNA/dose).

All treatments were administered by intravenous injection twice weekly (BIW) for 3 weeks. Tumor progression and metastasis to the liver were assessed by IVIS bioluminescence imaging on Days 14, 21, and 25. On Day 25, mice were euthanized, and a gross examination of the liver was performed to quantify liver metastasis and tumor burden across experimental groups.

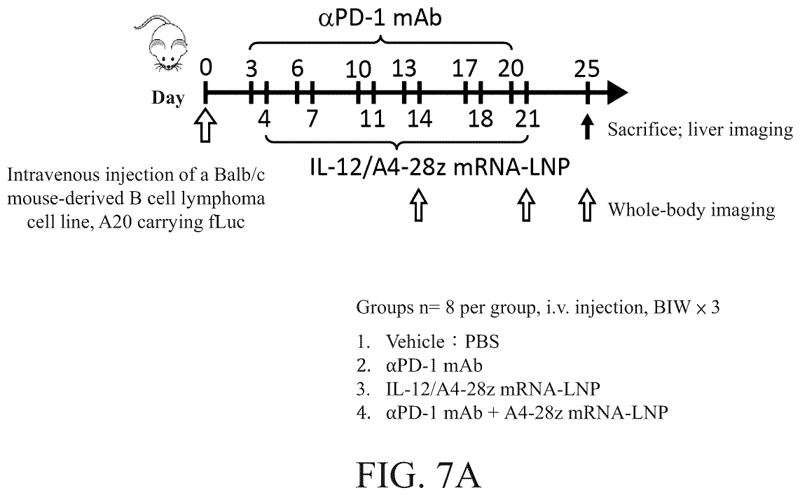

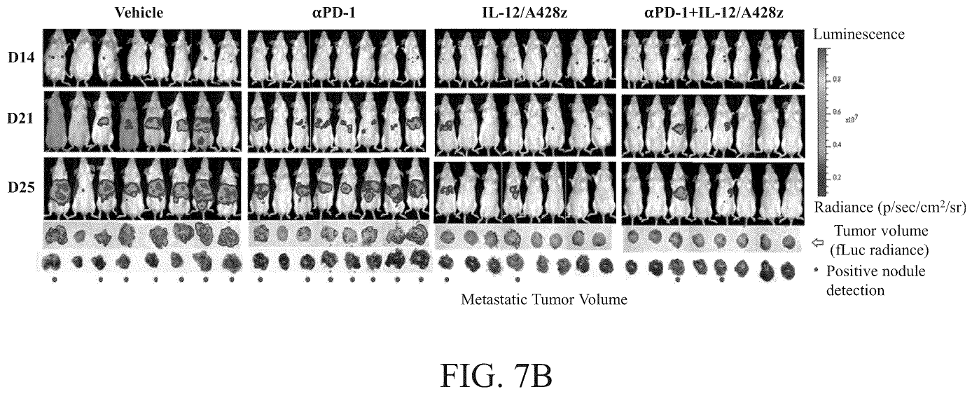

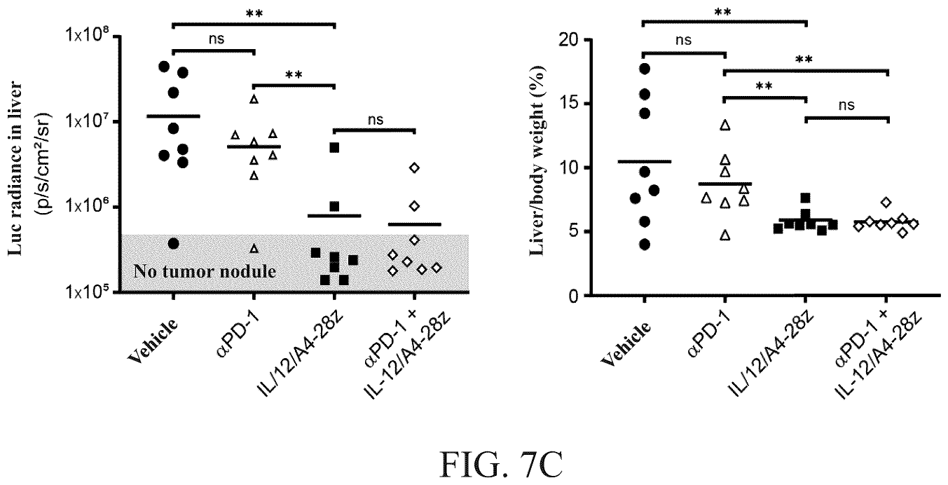

The results are shown in FIGS. 7B and 7C.

Mice treated with anti-PD-1 alone or vehicle control had no effect on tumor growth. On Day 25 (D25), mice treated with IL-12/A4-28z mRNA-LNP exhibited significant inhibition of tumor growth in liver compared to the anti-PD-1 alone or vehicle control group.

In order to further quantify liver metastasis, bioluminescence intensity in excised liver tissues was measured and the liver-to-body weight ratio was calculated (FIG. 7C). Both analyses yielded consistent results, showing that IL-12/A4-28z mRNA-LNP by itself can successfully prevented tumor metastasis to the liver, and anti-PD-1 antibody had no add-on effect on A20 tumor cell spreading and growth.

These findings demonstrate that IL-12/CD47-targeted CAR mRNA-LNP treatment, without the need of immune checkpoint blockade, can effectively inhibit disseminated tumor cell growth in liver.

While the invention has been described by way of example and in terms of the preferred embodiments, it should be understood that the invention is not limited to the disclosed embodiments. On the contrary, it is intended to cover various modifications and similar arrangements (as would be apparent to those skilled in the art). Therefore, the scope of the appended claims should be accorded the broadest interpretation so as to encompass all such modifications and similar arrangements.

What is claimed is:

1. An mRNA composition for treating cancer, comprising:
an mRNA encoding a CD47-targeted chimeric antigen receptor (CAR); and
an mRNA encoding interleukin-12 (IL-12).

2. The mRNA composition for treating cancer as claimed in claim 1, wherein the CD47-targeted chimeric antigen receptor comprises an extracellular domain, a transmembrane domain, and an intracellular domain, wherein the transmembrane domain is located between the extracellular domain and the intracellular domain and connects the extracellular domain to the intracellular domain.

3. The mRNA composition for treating cancer as claimed in claim 2, wherein the extracellular domain comprises a CD47 recognition domain, and the CD47 recognition domain comprises a single domain antibody fragment, and the single domain antibody fragment is capable of specifically binding to CD47.

4. The mRNA composition for treating cancer as claimed in claim 3, wherein the extracellular domain further comprises a hinge region, wherein the hinge region is connected to the transmembrane domain, and the hinge region comprises a CD28 derived hinge region.

5. The mRNA composition for treating cancer as claimed in claim 2, wherein the transmembrane domain comprises a CD28 derived transmembrane domain, and the intracellular domain comprises a CD28 derived intracellular T-cell costimulatory domain and a CD3ζ derived intracellular T-cell signaling domain.

6. The mRNA composition for treating cancer as claimed in claim 1, wherein the sequence of the mRNA encoding a CD47-targeted chimeric antigen receptor, from the 5′ end to the 3′ end, comprises an mRNA sequence encoding a CD47 recognition domain, an mRNA sequence encoding a CD28 derived intracellular T-cell costimulatory domain and an mRNA sequence encoding a CD3ζ derived intracellular T-cell signaling domain.

7. The mRNA composition for treating cancer as claimed in claim 1, wherein the interleukin-12 comprises a single chain interleukin-12.

8. The mRNA composition for treating cancer as claimed in claim 7, wherein the single chain interleukin-12 comprises a signal peptide, an interleukin-12 β subunit protein, and an interleukin-12 α subunit protein, and the sequence of the mRNA encoding interleukin-12, from the 5′ end to the 3′ end, comprises an mRNA sequence encoding the signal peptide, an mRNA sequence encoding the interleukin-12 β subunit protein and an mRNA sequence encoding the interleukin-12 α subunit protein.

9. The mRNA composition for treating cancer as claimed in claim 1, wherein the mRNA encoding a CD47-targeted chimeric antigen receptor and the mRNA encoding interleukin-12 are respectively located in two separate single strands.

10. The mRNA composition for treating cancer as claimed in claim 9, wherein a weight ratio of the mRNA encoding a CD47-targeted chimeric antigen receptor to the mRNA encoding interleukin-12 in the composition is 1-10:1-10.

11. The mRNA composition for treating cancer as claimed in claim 9, wherein the weight ratio of the mRNA encoding a CD47-targeted chimeric antigen receptor to the mRNA encoding interleukin-12 in the composition is 1:1 or 7:3.

12. A lipid nanoparticle preparation for treating cancer, comprising:
a lipid nanoparticle;
an mRNA encoding a CD47-targeted chimeric antigen receptor; and
an mRNA encoding interleukin-12,
wherein the lipid nanoparticle is loaded with the mRNA encoding a CD47-targeted chimeric antigen receptor and the mRNA encoding interleukin-12.

13. The lipid nanoparticle preparation for treating cancer as claimed in claim 12, wherein the sequence of the mRNA encoding a CD47-targeted chimeric antigen receptor, from the 5′ end to the 3′ end, comprises an mRNA sequence encoding a CD47 recognition domain, an mRNA sequence encoding a CD28 derived intracellular T-cell costimulatory domain and an mRNA sequence encoding a CD3ζ derived intracellular T-cell signaling domain.

14. The lipid nanoparticle preparation for treating cancer as claimed in claim 12, wherein the single chain interleukin-12 comprises a signal peptide, an interleukin-12 β subunit protein, and an interleukin-12 α subunit protein, and the sequence of the mRNA encoding interleukin-12, from the 5′ end to the 3′ end, comprises an mRNA sequence encoding the signal peptide, an mRNA sequence encoding the interleukin-12 β subunit protein, and an mRNA sequence encoding the interleukin-12 α subunit protein.

15. The lipid nanoparticle preparation for treating cancer as claimed in claim 12, wherein the lipid nanoparticle preparation for treating cancer comprises an injection dosage form.

16. A method for treating cancer, comprising:
administering an mRNA composition for treating cancer to a subject in need thereof,
wherein the mRNA composition for treating cancer comprises:
an mRNA encoding a CD47-targeted chimeric antigen receptor; and
an mRNA encoding interleukin-12;
wherein the mRNA composition for treating cancer is capable of polarizing an M2 macrophage into an M1 macrophage to secrete interleukin-12 and form a CD47-targeted chimeric antigen receptor-macrophage in a subject in need thereof.

17. The method for treating cancer as claimed in claim 16, wherein the sequence of the mRNA encoding a CD47-targeted chimeric antigen receptor, from the 5′ end to the 3′ end, comprises an mRNA sequence encoding a CD47 recognition domain, an mRNA sequence encoding a CD28 derived intracellular T-cell costimulatory domain and an mRNA sequence encoding a CD3ζ derived intracellular T-cell signaling domain.

18. The method for treating cancer as claimed in claim 16, wherein the single chain interleukin-12 comprises a signal peptide, an interleukin-12 β subunit protein, and an interleukin-12 α subunit protein, and the sequence of the mRNA encoding interleukin-12, from the 5′ end to the 3′ end, comprises an mRNA sequence encoding the signal peptide, an mRNA sequence encoding the interleukin-12 β subunit protein, and an mRNA sequence encoding the interleukin-12 α subunit protein.

19. The method for treating cancer as claimed in claim 16, wherein the cancer comprises a carcinoma in situ, an invasive carcinoma, or a metastatic cancer.

20. The method for treating cancer as claimed in claim 16, wherein the mRNA composition for treating cancer is a lipid nanoparticle preparation.
